SEQUENCE LISTING (59 sequences)

SEQ ID NO: 1 - protein, 14 aa, Artificial Sequence
  Chemicall synthesized
  misc_feature (1)..(2): Xaa can be any naturally occurring amino acid
  misc_feature (3)..(3): Xaa is Arg or Lys
  misc_feature (4)..(6): Xaa can be any naturally occurring amino acid
  misc_feature (8)..(10): Xaa can be any naturally occurring amino acid
  misc_feature (12)..(12): Xaa can be any naturally occurring amino acid
XXXXXXHXXXDXLA

SEQ ID NO: 2 - protein, 14 aa, Artificial Sequence
  Chemically synthesized
  misc_feature (1)..(1): Xaa can be any naturally occurring amino acid
  misc_feature (5)..(5): Xaa can be any naturally occurring amino acid
XTRRXLHRAFDTLA

SEQ ID NO: 3 - protein, 14 aa, Artificial Sequence
  Chemically synthesized
  misc_feature (2)..(2): Xaa can be any naturally occurring amino acid
  misc_feature (6)..(6): Xaa can be any naturally occurring amino acid
EXRRMXHRAFDTLA

SEQ ID NO: 4 - protein, 14 aa, Artificial Sequence
  Chemically synthesized
  misc_feature (8)..(8): Xaa can be any naturally occurring amino acid
  misc_feature (12)..(12): Xaa can be any naturally occurring amino acid
ETRRMLHXAFDXLA

SEQ ID NO: 5 - protein, 14 aa, Artificial Sequence
  Chemically synthesized
  misc_feature (1)..(1): Xaa can be any naturally occurring amino acid
  misc_feature (5)..(5): Xaa can be any naturally occurring amino acid
  misc_feature (8)..(8): Xaa can be any naturally occurring amino acid
  misc_feature (12)..(12): Xaa can be any naturally occurring amino acid
XTRRXLHXAFDXLA

SEQ ID NO: 6 - protein, 14 aa, Artificial Sequence
  Chemically synthesized
  misc_feature (1)..(1): Xaa can be any naturally occurring amino acid
  misc_feature (5)..(5): Xaa can be any naturally occurring amino acid
  misc_feature (12)..(12): Xaa can be any naturally occurring amino acid
XTRRXLHRAFDXLA

SEQ ID NO: 7 - protein, 14 aa, Artificial Sequence
  Chemically synthesized
  misc_feature (1)..(1): Xaa can be any naturally occurring amino acid
  misc_feature (5)..(5): Xaa can be any naturally occurring amino acid
  misc_feature (9)..(9): Xaa can be any naturally occurring amino acid
XERRXLHRXFDTLA

SEQ ID NO: 8 - protein, 14 aa, Artificial Sequence
  Chemically synthesized
  misc_feature (5)..(5): Xaa can be any naturally occurring amino acid
  misc_feature (12)..(12): Xaa can be any naturally occurring amino acid
ETRRXLHRAFDXLA

SEQ ID NO: 9 - protein, 14 aa, Artificial Sequence
  Chemically synthesized
  misc_feature (5)..(5): Xaa can be any naturally occurring amino acid
  misc_feature (9)..(9): Xaa can be any naturally occurring amino acid
ETRRXLHRXFDTLA

SEQ ID NO: 10 - protein, 14 aa, Artificial Sequence
  Chemically synthesized
  misc_feature (6)..(6): Xaa can be any naturally occurring amino acid
  misc_feature (10)..(10): Xaa can be any naturally occurring amino acid
ETRRMXHRAXDTLA

SEQ ID NO: 11 - protein, 14 aa, Artificial Sequence
  Chemically synthesized
  misc_feature (2)..(2): Xaa can be any naturally occurring amino acid
  misc_feature (9)..(9): Xaa can be any naturally occurring amino acid
EXRRMLHRXFDTLA

SEQ ID NO: 12 - protein, 14 aa, Artificial Sequence
  Chemically synthesized
  misc_feature (2)..(2): Xaa can be any naturally occurring amino acid
  misc_feature (6)..(6): Xaa can be any naturally occurring amino acid
  misc_feature (8)..(8): Xaa can be any naturally occurring amino acid
  misc_feature (12)..(12): Xaa can be any naturally occurring amino acid
EXRRMXHXAFDXLA

SEQ ID NO: 13 - protein, 16 aa, Artificial Sequence
  Chemically synthesized
  misc_feature (3)..(3): Xaa can be any naturally occurring amino acid
  misc_feature (7)..(7): Xaa can be any naturally occurring amino acid
AEXTRRXLHRAFDTLA

SEQ ID NO: 14 - protein, 12 aa, Artificial Sequence
  Chemically synthesized
  misc_feature (3)..(3): Xaa can be any naturally occurring amino acid
  misc_feature (10)..(10): Xaa can be any naturally occurring amino acid
RRXLHRAFDXLA

SEQ ID NO: 15 - protein, 13 aa, Artificial Sequence
  Chemically synthesized
  misc_feature (1)..(1): Xaa can be any naturally occurring amino acid
  misc_feature (5)..(5): Xaa can be any naturally occurring amino acid
XRRMXHRAFDTLA

SEQ ID NO: 16 - protein, 14 aa, Artificial Sequence
  Chemically synthesized
  misc_feature (5)..(5): Xaa can be any naturally occurring amino acid
  misc_feature (9)..(9): Xaa can be any naturally occurring amino acid
VTRRXLHRXFDTLA

SEQ ID NO: 17 - protein, 15 aa, Artificial Sequence
  Chemically synthesized
  misc_feature (6)..(6): Xaa can be any naturally occurring amino acid
  misc_feature (10)..(10): Xaa can be any naturally occurring amino acid
VVTRRXLHRXFDTLA

SEQ ID NO: 18 - protein, 14 aa, Artificial Sequence
  Chemically synthesized
  misc_feature (5)..(5): Xaa can be any naturally occurring amino acid
  misc_feature (9)..(9): Xaa can be any naturally occurring amino acid
RTRRXLHRXFDTLA

SEQ ID NO: 19 - protein, 15 aa, Artificial Sequence
  Chemically synthesized
  misc_feature (6)..(6): Xaa can be any naturally occurring amino acid
  misc_feature (10)..(10): Xaa can be any naturally occurring amino acid
RRTRRXLHRXFDTLA

SEQ ID NO: 20 - protein, 14 aa, Artificial Sequence
  Chemically synthesized
  misc_feature (5)..(5): Xaa can be any naturally occurring amino acid
  misc_feature (9)..(9): Xaa can be any naturally occurring amino acid
ATRAXLHRXFDTLA

SEQ ID NO: 21 - protein, 14 aa, Artificial Sequence
  Chemically synthesized
  misc_feature (5)..(5): Xaa can be any naturally occurring amino acid
  misc_feature (9)..(9): Xaa can be any naturally occurring amino acid
ATKAXLHRXFDTLA

SEQ ID NO: 22 - protein, 14 aa, Artificial Sequence
  Chemically synthesized
  misc_feature (6)..(6): Xaa can be any naturally occurring amino acid
  misc_feature (10)..(10): Xaa can be any naturally occurring amino acid
ATRAMXHRAXDTLA

SEQ ID NO: 23 - protein, 14 aa, Artificial Sequence
  Chemically synthesized
  misc_feature (1)..(1): Xaa is (S)-2-(4-pentenyl) alanine
  misc_feature (5)..(5): Xaa is (S)-2-(4-pentenyl) alanine
XTRRXLHRAFDTLA

SEQ ID NO: 24 - protein, 14 aa, Artificial Sequence
  Chemically synthesized
  misc_feature (2)..(2): Xaa is (S)-2-(4-pentenyl) alanine
  misc_feature (6)..(6): Xaa is (S)-2-(4-pentenyl) alanine
EXRRMXHRAFDTLA

SEQ ID NO: 25 - protein, 10 aa, Artificial Sequence
  Chemically synthesized
  misc_feature (1)..(1): Xaa is (S)-2-(4-pentenyl) alanine
  misc_feature (5)..(5): Xaa is (S)-2-(4-pentenyl) alanine
XTRRXLHRAF

SEQ ID NO: 26 - protein, 16 aa, Artificial Sequence
  Chemically synthesized
  misc_feature (3)..(3): Xaa is (S)-2-(4-pentenyl) alanine
  misc_feature (7)..(7): Xaa is (S)-2-(4-pentenyl) alanine
AEXTRRXLHRAFDTLA

SEQ ID NO: 27 - protein, 12 aa, Artificial Sequence
  Chemically synthesized
  misc_feature (3)..(3): Xaa is (S)-2-(4-pentenyl) alanine
  misc_feature (10)..(10): Xaa is (R)-2-(7-octenyl) alanine
RRXLHRAFDZLA

SEQ ID NO: 28 - protein, 13 aa, Artificial Sequence
  Chemically synthesized
  misc_feature (1)..(1): Xaa is (S)-2-(4-pentenyl) alanine
  misc_feature (5)..(5): Xaa is (S)-2-(4-pentenyl) alanine
XRRMXHRAFDTLA

SEQ ID NO: 29 - protein, 12 aa, Artificial Sequence
  Chemically synthesized
  misc_feature (4)..(4): Xaa is (S)-2-(4-pentenyl) alanine
  misc_feature (11)..(11): Xaa is (S)-2-(4-pentenyl) alanine
RRMXHRAFDTXA

SEQ ID NO: 30 - protein, 14 aa, Artificial Sequence
  Chemically synthesized
  misc_feature (5)..(5): Xaa is (S)-2-(4-pentenyl) alanine
  misc_feature (9)..(9): Xaa is (S)-2-(4-pentenyl) alanine
ETRRXLHRXFDTLA

SEQ ID NO: 31 - protein, 14 aa, Artificial Sequence
  Chemically synthesized
  misc_feature (5)..(5): Xaa is (S)-2-(4-pentenyl) alanine
  misc_feature (9)..(9): Xaa is (S)-2-(4-pentenyl) alanine
VTRRXLHRXFDTLA

SEQ ID NO: 32 - protein, 15 aa, Artificial Sequence
  Chemically synthesized
  misc_feature (6)..(6): Xaa is (S)-2-(4-pentenyl) alanine
  misc_feature (10)..(10): Xaa is (S)-2-(4-pentenyl) alanine
VVTRRXLHRXFDTLA

SEQ ID NO: 33 - protein, 14 aa, Artificial Sequence
  Chemically synthesized
  misc_feature (5)..(5): Xaa is (S)-2-(4-pentenyl) alanine
  misc_feature (9)..(9): Xaa is (S)-2-(4-pentenyl) alanine
RTRRXLHRXFDTLA

SEQ ID NO: 34 - protein, 15 aa, Artificial Sequence
  Chemically synthesized
  misc_feature (6)..(6): Xaa is (S)-2-(4-pentenyl) alanine
  misc_feature (10)..(10): Xaa is (S)-2-(4-pentenyl) alanine
RRTRRXLHRXFDTLA

SEQ ID NO: 35 - protein, 14 aa, Artificial Sequence
  Chemically synthesized
  misc_feature (5)..(5): Xaa is (S)-2-(4-pentenyl) alanine
  misc_feature (9)..(9): Xaa is (S)-2-(4-pentenyl) alanine
ATRAXLHRXFDTLA

SEQ ID NO: 36 - protein, 14 aa, Artificial Sequence
  Chemically synthesized
  misc_feature (5)..(5): Xaa is (S)-2-(4-pentenyl) alanine
  misc_feature (9)..(9): Xaa is (S)-2-(4-pentenyl) alanine
ATKAXLHRXFDTLA

SEQ ID NO: 37 - protein, 14 aa, Artificial Sequence
  Chemically synthesized
  misc_feature (6)..(6): Xaa is (S)-2-(4-pentenyl) alanine
  misc_feature (10)..(10): Xaa is (S)-2-(4-pentenyl) alanine
ATRAMXHRAXDTLA

SEQ ID NO: 38 - protein, 14 aa, Artificial Sequence
  Chemically synthesized
  misc_feature (8)..(8): Xaa is (S)-2-(4-pentenyl) alanine
  misc_feature (12)..(12): Xaa is (S)-2-(4-pentenyl) alanine
ETRRMLHXAFDXLA

SEQ ID NO: 39 - protein, 14 aa, Artificial Sequence
  Homo sapiens
ETRRMLHRAFDTLA

SEQ ID NO: 40 - protein, 20 aa, Artificial Sequence
  Chemically synthesized
  misc_feature (11)..(11): Xaa can be any naturally occurring amino acid
  misc_feature (15)..(15): Xaa can be any naturally occurring amino acid
AGATAEETRRXLHRXFDTLA

SEQ ID NO: 41 - protein, 25 aa, Artificial Sequence
  Chemically synthesized
  misc_feature (16)..(16): Xaa can be any naturally occurring amino acid
  misc_feature (20)..(20): Xaa can be any naturally occurring amino acid
FGAVGAGATAEETRRXLHRXFDTLA

SEQ ID NO: 42 - protein, 14 aa, Artificial Sequence
  Chemically synthesized
  misc_feature (5)..(5): Xaa can be any naturally occurring amino acid
  misc_feature (9)..(9): Xaa can be any naturally occurring amino acid
KTRRXLHRXFDTLA

SEQ ID NO: 43 - protein, 14 aa, Artificial Sequence
  Chemically synthesized
  misc_feature (5)..(5): Xaa can be any naturally occurring amino acid
  misc_feature (9)..(9): Xaa can be any naturally occurring amino acid
QTRRXLHRXFDTLA

SEQ ID NO: 44 - protein, 14 aa, Artificial Sequence
  Chemically synthesized
  misc_feature (5)..(5): Xaa can be any naturally occurring amino acid
  misc_feature (9)..(9): Xaa can be any naturally occurring amino acid
NTRRXLHRXFDTLA

SEQ ID NO: 45 - protein, 16 aa, Artificial Sequence
  Chemically synthesized
  misc_feature (7)..(7): Xaa can be any naturally occurring amino acid
  misc_feature (11)..(11): Xaa can be any naturally occurring amino acid
VVVTRRXLHRXFDTLA

SEQ ID NO: 46 - protein, 14 aa, Artificial Sequence
  Chemically synthesized
  misc_feature (5)..(5): Xaa can be any naturally occurring amino acid
  misc_feature (9)..(9): Xaa can be any naturally occurring amino acid
ITRRXLHRXFDTLA

SEQ ID NO: 47 - protein, 14 aa, Artificial Sequence
  Chemically synthesized
  misc_feature (5)..(5): Xaa can be any naturally occurring amino acid
  misc_feature (9)..(9): Xaa can be any naturally occurring amino acid
LTRRXLHRXFDTLA

SEQ ID NO: 48 - protein, 14 aa, Artificial Sequence
  Chemically synthesized
  misc_feature (5)..(5): Xaa can be any naturally occurring amino acid
  misc_feature (9)..(9): Xaa can be any naturally occurring amino acid
FTRRXLHRXFDTLA

SEQ ID NO: 49 - protein, 14 aa, Artificial Sequence
  Chemically synthesized
  misc_feature (5)..(5): Xaa can be any naturally occurring amino acid
  misc_feature (9)..(9): Xaa can be any naturally occurring amino acid
WTRRXLHRXFDTLA

SEQ ID NO: 50 - protein, 14 aa, Artificial Sequence
  Chemically synthesized
  misc_feature (5)..(5): Xaa can be any naturally occurring amino acid
  misc_feature (9)..(9): Xaa can be any naturally occurring amino acid
YTRRXLHRXFDTLA

SEQ ID NO: 51 - DNA, 38 bp, Artificial Sequence
  Chemically synthesized
gacaagcttgcgatgttttccggtggcggcggcccgct

SEQ ID NO: 52 - DNA, 36 bp, Artificial Sequence
  Chemically synthesized
gtgtctagatcatgctagagtatcaaaggctctatg

SEQ ID NO: 53 - DNA, 35 bp, Artificial Sequence
  Chemically synthesized
gtcaagcttgggatgacggattcccctggcggtgt

SEQ ID NO: 54 - DNA, 36 bp, Artificial Sequence
  Chemically synthesized
gactctagatcaggggaatccaaaaccatacggggt

SEQ ID NO: 55 - DNA, 19 bp, Artificial Sequence
  Chemically synthesized
acatcatccctgcctctac

SEQ ID NO: 56 - DNA, 18 bp, Artificial Sequence
  Chemically synthesized
tcaaaggtggaggagtgg

SEQ ID NO: 57 - protein, 1235 aa, Artificial Sequence
  Homo sapiens
MFSGGGGPLSPGGKSAARAASGFFAPAGPRGASRGPPPCLRQNFYNPYLAPVGTQQKPTGPTQRHTYYSECDEFRFIAPRVLDEDAPPEKRAGVHDGHLKRAPKVYCGGDERDVLRVGSGGFWPRRSRLWGGVDHAPAGFNPTVTVFHVYDILENVEHAYGMRAAQFHARFMDAITPTGTVITLLGLTPEGHRVAVHVYGTRQYFYMNKEEVDRHLQCRAPRDLCERMAAALRESPGASFRGISADHFEAEVVERTDVYYYETRPALFYRVYVRSGRVLSYLCDNFCPAIKKYEGGVDATTRFILDNPGFVTFGWYRLKPGRNNTLAQPAAPMAFGTSSDVEFNCTADNLAIEGGMSDLPAYKLMCFDIECKAGGEDELAFPVAGHPEDLVIQISCLLYDLSTTALEHVLLFSLGSCDLPESHLNELAARGLPTPVVLEFDSEFEMLLAFMTLVKQYGPEFVTGYNIINFDWPFLLAKLTDIYKVPLDGYGRMNGRGVFRVWDIGQSHFQKRSKIKVNGMVNIDMYGIITDKIKLSSYKLNAVAEAVLKDKKKDLSYRDIPAYYAAGPAQRGVIGEYCIQDSLLVGQLFFKFLPHLELSAVARLAGINITRTIYDGQQIRVFTCLLRLADQKGFILPDTQGRFRGAGGEAPKRPAAAREDEERPEEEGEDEDEREEGGGEREPEGARETAGRHVGYQGARVLDPTSGFHVNPVVVFDFASLYPSIIQAHNLCFSTLSLRADAVAHLEAGKDYLEIEVGGRRLFFVKAHVRESLLSILLRDWLAMRKQIRSRIPQSSPEEAVLLDKQQAAIKVVCNSVYGFTGVQHGLLPCLHVAATVTTIGREMLLATREYVHARWAAFEQLLADFPEAADMRAPGPYSMRIIYGDTDSIFVLCRGLTAAGLTAVGDKMASHISRALFLPPIKLECEKTFTKLLLIAKKKYIGVIYGGKMLIKGVDLVRKNNCAFINRTSRALVDLLFYDDTVSGAAAALAERPAEEWLARPLPEGLQAFGAVLVDAHRRITDPERDIQDFVLTAELSRHPRAYTNKRLAHLTVYYKLMARRAQVPSIKDRIPYVIVAQTREVEETVARLAALRELDAAAPGDEPAPPAALPSPAKRPRETPSPADPPGGASKPRKLLVSELAEDPAYAIAHGVALNTDYYFSHLLGAACVTFKALFGNNAKITESLLKRFIPEVWHPPDDVAARLRTAGFGAVGAGATAEETRRMLHRAFDTLA

SEQ ID NO: 58 - protein, 488 aa, Artificial Sequence
  Homo sapiens
MTDSPGGVAPASPVEDASDASLGQPEEGAPCQVVLQGAELNGILQAFAPLRTSLLDSLLVMGDRGILIHNTIFGEQVFLPLEHSQFSRYRWRGPTAAFLSLVDQKRSLLSVFRANQYPDLRRVELAITGQAPFRTLVQRIWTTTSDGEAVELASETLMKRELTSFVVLVPQGTPDVQLRLTRPQLTKVLNATGADSATPTTFELGVNGKFSVFTTSTCVTFAAREEGVSSSTSTQVQILSNALTKAGQAAANAKTVYGENTHRTFSVVVDDCSMRAVLRRLQVGGGTLKFFLTTPVPSLCVTATGPNAVSAVFLLKPQKICLDWLGHSQGSPSAGSSASRASGSEPTDSQDSASDAVSHGDPEDLDGAARAGEAGALHACPMPSSTTRVTPTTKRGRSGGEDARADTALKKPKTGSPTAPPPADPVPLDTEDDSDAADGTAARPAAPDARSGSRYACYFRDLPTGEASPGAFSAFRGGPQTPYGFGFP

SEQ ID NO: 59 - protein, 15 aa, Artificial Sequence
  Chemically synthesized
VVTRRCLHRCFDTLA

(19) United States
(12) Patent Application Publication
(10) Pub. No.: US 2023/0235301 A1
(43) Pub. Date: Jul. 27, 2023

(54) Compounds and Methods for Treating, Ameliorating, or Preventing Herpes Ocular Keratitis

(71) Applicant: THE TRUSTEES OF THE UNIVERSITY OF PENNSYLVANIA, Philadelphia, PA (US)

(72) Inventors: Robert P. Ricciardi, Kennett Square, PA (US); Manunya Nuth, Green Lane, PA (US); Hancheng Guan, Downingtown, PA (US); Richard W. Scott, West Chester, PA (US); Michael H. Parker, Chalfont, PA (US); Allen B. Reitz, Lansdale, PA (US)

(21) Appl. No.: 18/000,644

(22) PCT Filed: Jun. 3, 2021
(86) PCT No.: PCT/US2021/035726

§ 371 Date: Dec. 2, 2022

Related U.S. Application Data
(60) Provisional application No. 63/034,660, filed on Jun. 4, 2020.

(51) Int. Cl. C12N 9/12; A61P 31/22; A61K 38/45; A61K 45/06; A61K 9/127

(52) U.S. Cl. CPC C12N 9/1252; A61K 9/1271; A61K 38/45; A61K 45/06; A61P 31/22; C12Y 207/07007

(57) ABSTRACT

The present disclosure relates generally to stapled peptides, and pharmaceutical compositions thereof, which are useful for preventing and/or treating herpes simplex virus-1 (HSV-1) processive DNA synthesis, propagation, and/or infection in a subject. The present disclosure further provides methods for treating herpes simplex keratitis in a subject

### CROSS-REFERENCE TO RELATED APPLICATION

The present application claims priority under 35 U.S.C. § 119(e) to U.S. Provisional Pat. Application No. 63/034,660, filed Jun. 4, 2020, which is incorporated herein by reference in its entirety.

### STATEMENT REGARDING FEDERALLY SPONSORED RESEARCH OR DEVELOPMENT

This invention was made with government support under EY026849 awarded by the National Institutes of Health. The government has certain rights in the invention.

### SEQUENCE LISTING

The ASCII text file named “046483-7265WO1(02581) Sequence Listing,” created on Jun. 4, 2021, comprising 38.4 Kbytes, is hereby incorporated by reference in its entirety.

### BACKGROUND

Herpes simplex virus type 1 (HSV-1) is a ubiquitous pathogen that spreads through saliva and nasal secretions and is capable of causing a range of ocular pathologies in the cornea, conjunctiva, uvea, and retina. Following the initial infection, HSV-1 enters nerves endings and subsequently migrates to ganglionic nuclei such as the cranial trigeminal ganglia (TG). Over time, HSV-1 DNA becomes stably maintained within the TG and enters into a non-infectious latent state. Latent HSV-1 will persist indefinitely, and upon physiological triggers such as fatigue or hormones will become reactivated. Following reactivation, HSV-1 can migrate down the mandibular branch of the TG and manifest as a cold sore. Alternatively, the reactivated HSV-1 can migrate along the ophthalmic branch of the TG where it will infect the eye, resulting in Herpes Keratitis (HK).

Reactivation of HSV-1 from the ophthalmic branch of the TG results in two classic types of corneal pathologies. Epithelial Keratitis presents as lesions restricted to the outer epithelium, caused by the destruction of replicating virus. They begin as vesicular eruptions in the corneal epithelium, but quickly coalesce into dendritic shaped lesions, causing blurred vision, photophobia, pain and sensation of a foreign body in one or both eyes, tearing, and/or redness. The lesions can progress to enlarged, non-linear (geographic) lesions, especially if corticosteroids are used in the treatment. In humans, these lesions can heal rapidly if treated with antiviral drugs, mainly acyclovir, which remains the gold standard of care despite the recent increase in drug-resistant mutants. Stromal Keratitis can develop from infectious Epithelial Keratitis involving retrograde (reverse) migration of virus from the epithelium to the ophthalmic branch of the TG followed by anterograde (forward) migration to the corneal stroma. Stromal Keratitis clinical signs include stromal opacity, disc-shaped edema, and localized inflammation. In addition to the damaging effects of the infectious virus, there is a significant immune response to viral proteins. This promotes the ingrowth of blood vessels and infiltration of leukocytes, causing damage to the corneal stroma. Recurrent infections culminate in scarring of the cornea and account for the greatest loss of vision and blindness. If left untreated, HK may lead to permanent corneal scarring, thinning, opacification, and neovascularization, with loss of vision, leaving corneal transplantation as the only option for restoration of sight. However, there is still risk of reactivated latent infection affecting the transplanted cornea. HK is the leading cause of both cornea-derived and infection-associated blindness in the developed world: about 500,000 cases in the U.S. The recurrence rate of HK is ~27% at 1 year, 50% at 5 years and 63% at 20 years.

Clinical management of HSV infections largely relies on the use of nucleoside analogue antiviral drugs. The gold standard for treatment is the anti-herpetic agent acyclovir (ACV). ACV blocks HSV-1 infection by targeting viral thymidine kinase (TK). Clearly, ACV is extremely effective against oral and genital herpes with negligible drug failures. Strikingly by contrast, the emergence of viral resistant mutants in immune-competent HK patients is about 7% and even greater in immune-compromised HK patients. This substantial level of ACV resistance is apparently related to the immunologically privileged status of the eye. Significantly, the mechanism of drug resistance correlates directly with specific mutations in the TK gene of HSV-1 isolated from HK patients unresponsive to ACV. None of the other ACV-related inhibitors (such as valacyclovir, famciclovir, ganciclovir, vidarabine) can be used when HK becomes unresponsive to ACV because they are all directed against the same target (herpes TK). This leaves only trifluridine as an FDA approved drug against ocular HK. While trifluridine (Viroptic) has proven effectiveness, its prolonged use is limited by concerns of toxicity because it does not specifically target HSV-1 and can block any DNA polymerase. This complicates the clinical management of difficult and refractory cases.

Therefore, there is a need in the art for novel compounds and methods for the treatment, amelioration, and/or prevention of HK. The present disclosure addresses and meets this unmet need.

### BRIEF SUMMARY OF THE DISCLOSURE

In one aspect, the present disclosure provides a stapled peptide of formula (I):
Xaa1-Xaa2-Xaa3-Xaa4-Xaa5-Xaa6-Xaa7-Xaa8-Xaa9-Xaa10-Xaa11-Xaa12-Xaa13-Xaa14 (I), SEQ ID NO:1,
wherein the residues Xaa1-Xaa14 are defined as follows:
   Xaa3 is Arg or Lys; Xaa7 is His; Xaa11 is Asp; Xaa13 is Leu; and Xaa14 is Ala;
   at least one residue pair selected from Xaa1-Xaa5, Xaa2-Xaa6, Xaa2-Xaa9, Xaa5-Xaa9, Xaa5-Xaa12, Xaa6-Xaa10, and Xaa8-Xaa12 is a residue pair in which α-carbons are covalently linked through an independently selected linker; and
   the remaining residues selected from Xaa1, Xaa2, Xaa4, Xaa5, Xaa6, Xaa8, Xaa9, Xaa10, and Xaa12 are naturally occurring amino acids, wherein Xaa1 can be absent or Xaa1-Xaa2 can be absent; or a salt or solvate thereof, wherein the linker is defined elsewhere herein.

In another aspect, the present disclosure further provides methods of preventing, ameliorating, and/or treating herpes simplex virus-1 (HSV-1) processive DNA synthesis, propagation, and/or infection in a subject by administering to the subject a therapeutically effective amount of a compound of the present disclosure. In certain embodiments, the present disclosure provides a method of treating, ameliorating and/or preventing herpes keratitis in a subject by administering to the subject a therapeutically effective amount of a compound of the present disclosure.

In yet another aspect, the present disclosure provides a kit comprising a compound of the present disclosure, further comprising an applicator; and an instructional material for the use of the kit, wherein the instruction material comprises instructions for treating, ameliorating, and/or preventing herpes keratitis in a subject.

In yet another aspect, the present disclosure provides a pharmaceutical composition comprising a compound of the present disclosure and a pharmaceutically acceptable carrier. In certain embodiments, the pharmaceutically acceptable carrier comprises liposomes. In certain embodiments, the liposomes are coated with chitosan.

### BRIEF DESCRIPTION OF THE DRAWINGS

The following detailed description of specific embodiments of the present disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1 illustrates the eye of a subject suffering from HK.

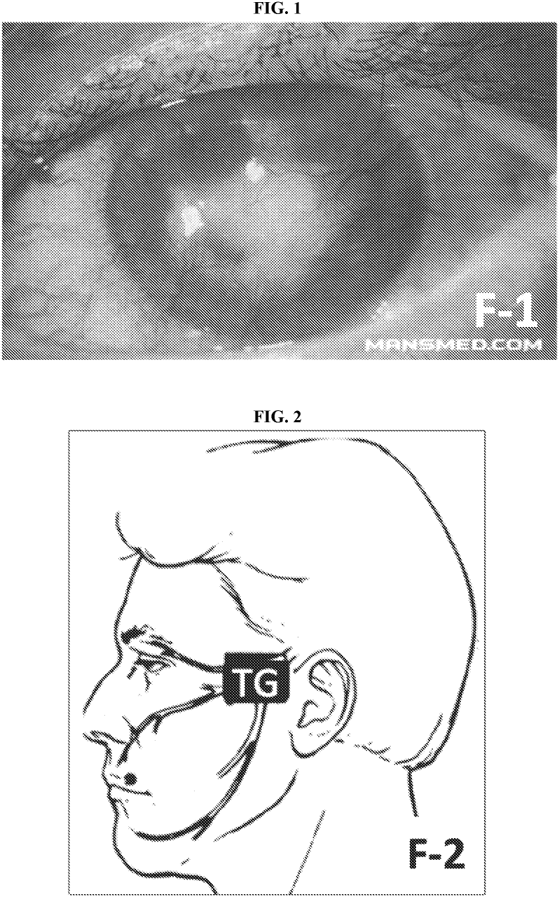

FIG. 2 illustrates migration of herpes virus along the branches of the cranial trigeminal ganglia (TG).

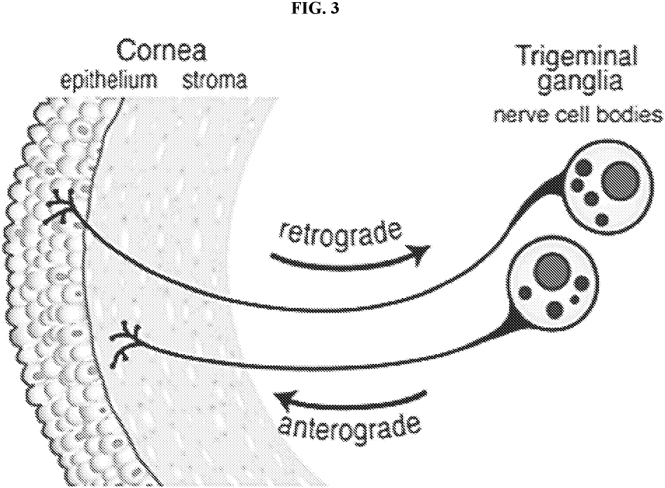

FIG. 3 illustrates how herpes infection spreads in retrograde fashion from the cornea epithelium to the trigeminal (TG) ganglia, followed by anterograde spread to the corneal stroma.

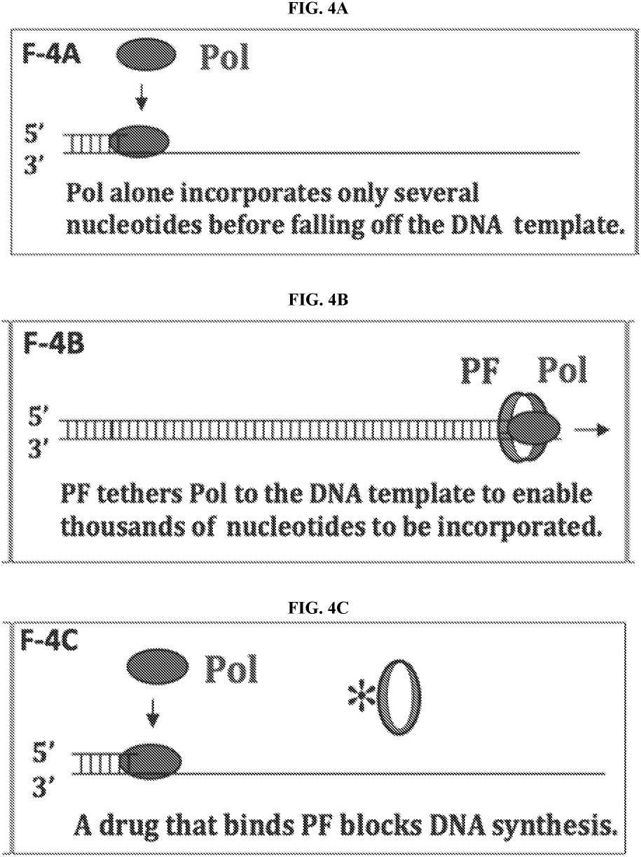

FIGS. 4A-4C illustrate certain interactions between DNA polymerases and processivity factors.

FIG. 5 illustrates a non-limiting stapled α-helical peptide.

FIG. 6 illustrates the interaction between HSV-1 DNA Polymerase (UL30) and Processivity Factor (UL42). The extreme C-terminus of UL30 forms an α-helix (shown as a ribbon with residues indicated) that makes multiple interactions with UL42. Hydrogen bonds between three residues (K289, R64, and D63) of UL42 with residues R1224, H1228, D1232, L1234, and A1235 (boxed) of UL30 C-terminus are indicated as dotted lines. This interactive depiction is based on the UL30/UL42 co-crystal structure of HSV-1.

FIG. 7 provides non-limiting examples of stapled peptide constructs of the present disclosure: N-pep (SEQ ID NO:39), S-pep1 (SEQ ID NO:23), S-pep2 (SEQ ID NO:25), S-pep3 (SEQ ID NO:26), S-pep4 (SEQ ID NO:27), S-pep5 (SEQ ID NO:28), S-pep6 (SEQ ID NO:29), S-pep7 (SEQ ID NO:30), and S-pep8 (SEQ ID NO:38). Stapled peptide constructs based on the sequence of the extreme C-terminus of HSV-1 UL30 DNA polymerase. The native peptide (N-pep) comprises the C-terminal 14 amino acid residues 1222 to 1235 of UL30 (SEQ ID NO:57). The amino acids of UL30 peptides that are known to make contact with HSV-1 Processivity Factor (UL42) (SEQ ID NO: 58) are boxed. Stapled peptides (S-pep1 to S-pep8) were generated by all-hydrocarbon crosslinking at positions i, i+4 or i, i+7. X = (S)-2-(4-pentenyl) alanine; X/R8 = (R)-2-(7-octenyl) alanine.

FIG. 8 illustrates a subset of stapled peptides: NATIVE (SEQ ID NO:39), SPep1 (SEQ ID NO:2), S1P* (SEQ ID NO:3), S2P (SEQ ID NO:4), S3P (SEQ ID NO:5), S4P (SEQ ID NO:6), S5P (SEQ ID NO:7), S6P (SEQ ID NO:8), S7P (SEQ ID NO:9), S8P (SEQ ID NO:10), S9P (SEQ ID NO:11), and S10P (SEQ ID NO:12). The native peptide is shown at the top, and the contact residues are shaded.

FIG. 9 illustrates synthesis of non-limiting stitched peptides (with two staples) (SEQ ID NO:6).

FIG. 10 illustrates synthesis of non-limiting triazole-stapled peptides (SEQ ID NO:2).

FIGS. 11A-11F provide graphs showing inhibition of HSV-1 processive DNA synthesis by stapled peptides in a mechanistic in vitro DNA processivity assay. HSV-1 UL30 DNA Polymerase and UL42 Processivity Factor were translated in vitro and then used to conduct processive DNA synthesis in the presence of increasing amounts of Native (N) or Stapled (S) peptides. DNA synthesis was quantified via product-dependent colorimetry. The data represents mean ± SD from at least two independent experiments in triplicate. FIG. 11A: Native peptide (N-pep); FIG. 11B: S-pep1; FIG. 11C: S-pep3; FIG. 11D: S-pep4; FIG. 11E: S-pep7; FIG. 11F: S-pep8.

FIG. 12 illustrates the fact that the 36C of C-Pol (UL30) forms an αβα structure when co-crystallized with PF (UL42), and the fact that certain stapled peptides of the disclosure are derived from Helix-2, which is the extreme C-terminus of C-Pol.

FIGS. 13A-13C illustrate three stapled peptides: S-pep1 (FIG. 13A); S-pep7 (FIG. 13B); and S-pep7B (FIG. 13C).

FIG. 14 illustrates an IC₅₀ plate assay for quantitating processive DNA synthesis in vitro.

FIGS. 15A-15F provide graphs showing inhibition of HSV-1 plaque formation by stapled peptides in Vero cells. HSV-1 (~100 PFU) was first absorbed onto near confluent Vero cells for 1 h to allow cell attachment and entry of the virus. After the absorption, increasing concentrations of peptides were added. Following 55 h treatment, viral plaques were counted and used to calculate EC₅₀ values. FIG. 15A: Native peptide (N-pep); FIG. 15B: S-pep1; FIG. 15C: S-pep3; FIG. 15D: S-pep4; FIG. 15E: S-pep7; FIG. 15F: S-pep8.

FIG. 16 provides the sequence of amino acids derived from amino acid substitutions or additions introduced at the N-terminus of S-pep7. The N-terminal Glu¹²²² in S-pep7 (SEQ ID NO:9) was replaced by a single Val (S-pep7A, SEQ ID NO:16), two Val (S-pep7B, SEQ ID NO: 17), a single Arg (S-pep7C, SEQ ID NO: 18), or two Arg (S-pep7D, SEQ ID NO: 19). All altered amino acid residues, with respect to S-pep7, are italicized and underlined.

FIGS. 17A-17D show in vitro processive DNA synthesis conducted by recombinant proteins of HSV-1, UL42, and UL30 in the presence of increasing concentrations of each modified S-peptide: S-pep7A (FIG. 17A); S-pep7B (FIG. 17B); S-pep7C (FIG. 17C); S-pep7D (FIG. 17D).

FIGS. 18A-18C provide the results of HSV-1 plaque reduction assays. Following 1 h absorption of HSV-1 onto Vero cells, S-pep7A (FIG. 18A) and S-pep7B (FIG. 18B) were added at increasing concentrations and plaques were counted after 55 h. For direct comparison, inhibition of HSV-1 plaques by Acyclovir (ACV) was also performed (FIG. 18C).

FIG. 19 shows the results of cytotoxicity studies using S-pep7B. Vero cells were treated with S-pep7B at two-fold serial dilutions for 24 h and measured for intracellular ATP content and LDH leakage. Data represents mean ± SD obtained from at least two independent experiments performed in triplicate.

FIGS. 20A-20B show the specificity of S-pep7B for HSV-1 by demonstrating the inability of S-pep7B to block both in vitro processive DNA synthesis conducted by vaccinia virus proteins (FIG. 20A) and vaccinia virus infection (FIG. 20B). The data represents mean ± SD from at least two independent experiments performed in duplicate.

FIG. 21 shows inhibition of HSV-1 DNA replication by S-pep7B in infected cells. Confluent Vero cells were infected by absorbing HSV-1 (MOI ~1) for 1 h (marked as time point 0 h), followed by treatment with vehicle DMSO or S-pep7B at the indicated concentrations. At 4 h post-treatment, viral genomic DNA was extracted from the cells and then used for amplification of the UL42 gene. Following agarose gel electrophoresis, relative % levels of UL42 DNA were determined after being normalized to that of the cellular house-keeping gene GAPDH. The level of UL42 DNA from vehicle treatment was arbitrarily set at 100.

FIG. 22A: inhibition of HSV-1 infection of human primary corneal epithelial cells by S-pep7B. Following 1 h absorption of HSV-1 onto human primary corneal epithelial cells, the cells were washed thoroughly to remove any unabsorbed virus and then treated with S-pep7B at different dilutions. At 72 h post-treatment, virus titers in the culture media were determined by plaque reduction assays using Vero cells. FIG. 22B: S-pep7B cytotoxicity in the primary cells was determined after 24 h by measuring ATP content. All data represent mean ± SD obtained from two independent experiments in duplicate.

FIGS. 23A-23C show HSV-1 infection in human organotypic (3D) corneal epithelial cultures is blocked by S-pep7B. FIG. 23A: Hematoxylin and eosin (H&E) photomicrograph of cross section of corneal organotypic culture with arrow pointing to corneal epithelial layer on top of fibroblasts (asterisk). FIG. 23B: HSV-1 infection of corneal organotypic culture is blocked by topical application of S-pep7B at 25 µM. FIG. 23C: Immunostaining of a representative cross section of HSV-1 infected 3D organotypic corneal culture treated with 25 µM S-pep7B revealed absence of green stain for detecting viral structural protein gB, due to inhibition of infection, as well as an unremarkable nuclear morphology upon DAPI staining.

FIG. 24: Chitosan-coated liposomes formulated for delivery of S-pep7B into human corneal 3D epithelial cells. Following cell membrane penetration, S-pep7B diffuses out of the chitosan-coated liposomes into the cytoplasm.

FIG. 25 shows the chemical structure of cyclic cell penetrating peptide CPP9.

FIG. 26 shows CPP conjugates linked through either N-terminal or C-terminal ends (SEQ ID NO: 17).

FIG. 27 shows a non-limiting synthetic route for attachment of CPP9 to a stapled peptide via a cysteine-crosslinking staple (SEQ ID NO:59).

FIG. 28 provides non-limiting examples of peptides with additional N-terminal amino acid residues with increased hydrophobicity, polarity, and/or cationic charge.

### DETAILED DESCRIPTION

The present disclosure relates in one aspect to the identification of certain compounds that block HSV-1′s processivity factor (PF) from engaging with HSV-1′s DNA polymerase. Without the PF, the HSV-1′s polymerase is unable to replicate the viral DNA, thereby blocking HSV-1′s propagation. As described elsewhere herein, the compounds of the disclosure bind to certain regions of HSV-1′s PF, thereby making the PF inaccessible to the HSV-1 polymerase. In certain embodiments, the compounds of the disclosure inhibit processive DNA synthesis and HSV-1 infection in mammalian cells.

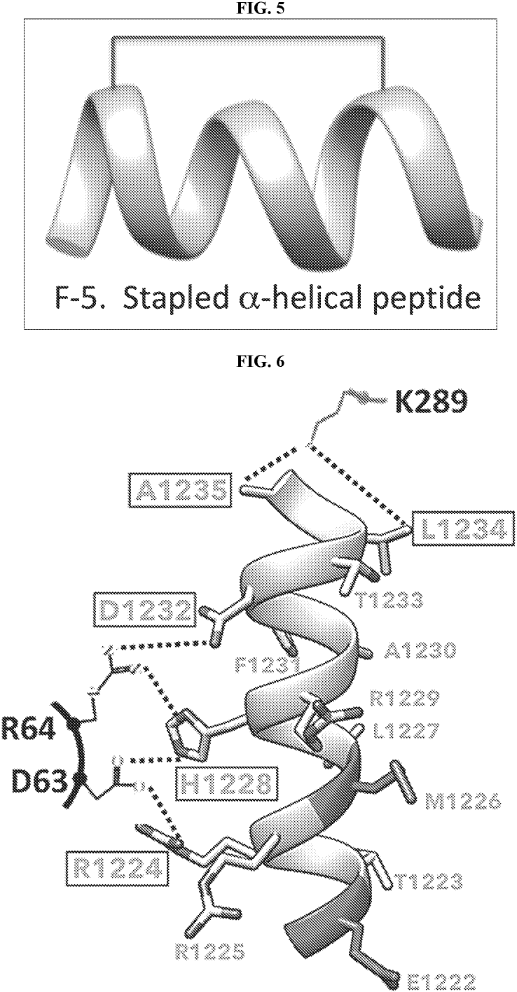

Processivity Factors (PFs) are essential for viral propagation and serve as new drug targets. DNA polymerases (Pols), from viruses to mammals, fail to synthesize extended chains in the absence of Processivity Factors (PFs). Each PF functions only with its cognate Pol. In the case of viral PFs, there are no cellular homologues, making them specific drug targets. Catalytic efficiency of DNA Pols requires that they function processively, i.e., they must be capable of incorporating nucleotides continuously without dissociating from the template. The mechanism by which DNA Pols achieve catalytic efficiency is through associating with their PFs, and this association tethers them to the DNA such that the rate of Pol nucleotide incorporation exceeds the rate of Pol dissociation from the template (FIGS. 4A-4B). For example, the DNA Pol of KSHV (Kaposi’s Sarcoma Human Virus) singly incorporates only 3 nucleotides; however, in the presence of its PF, it incorporates many thousands of nucleotides. The PFs are fittingly referred to as sliding clamps, and have been identified in Human Herpes-6, KSHV, Molluscum Contagiosum Virus (MCV), Feline Herpes Virus (FHV-1), and Vaccinia Virus (Smallpox virus).

As depicted in FIG. 4C, a small chemical inhibitor that binds to PF can be used to disable DNA synthesis. In certain embodiments, certain small molecule compounds can be developed to directly bind to their respective PF target proteins, and thus block processive DNA synthesis in vitro and block cellular infection. However, small molecules that block HSV-1 processivity in vitro often lack sufficient potencies prompting a paradigm shift and raising questions about the use of these small molecule compounds to treat HK.

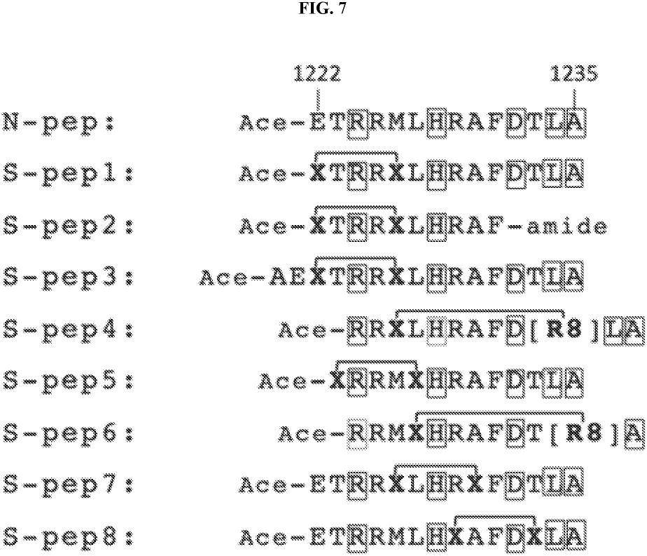

As demonstrated herein, certain stapled α-helical peptides can be used as therapeutics for HK. Stapled α-helical peptides can act as inhibitors of protein-protein interactions (FIG. 5). Without wishing to be limited by any theory, incorporating macrocyclic links or staples into α-helical peptides imparts a rigid conformation to the α-helical peptides and enables the formation of stable contacts to the target protein. In certain embodiments, unlike natural peptides, stapling the α-helix also creates a protease shield that extends residence time of the α-helix in the cell. In certain embodiments, the stapled peptides of the disclosure engage a unique target and block a known mechanism of action required for viral replication. Due to their specificity, stapled peptides of the disclosure provide a superior means of disrupting protein-protein interactions with minimal off-target effects.

### Definitions

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, the preferred methods and materials are described.

Generally, the nomenclature used herein and the laboratory procedures in cell culture, molecular biology, organic chemistry, virology, and nucleic acid chemistry and hybridization are those well-known and commonly employed in the art. The nomenclature used herein and the laboratory procedures used in analytical chemistry described below are those well-known and commonly employed in the art. Standard techniques or modifications thereof, are used for chemical syntheses and chemical analyses.

Standard techniques are used for peptide synthesis. The techniques and procedures are generally performed according to conventional methods in the art and various general references (e.g., Sambrook and Russell, 2012, Molecular Cloning, A Laboratory Approach, Cold Spring Harbor Press, Cold Spring Harbor, NY, and Ausubel et al., 2002, Current Protocols in Molecular Biology, John Wiley & Sons, NY), which are provided throughout this document.

As used herein, each of the following terms has the meaning associated with it in this section.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

As used herein, “about” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the term “acylated” refers to the chemical moiety —C(═O)R, wherein R is optionally substituted C₁-C₂₀ alkyl, C₃-C₈ cycloalkyl, aryl, heteroaryl, or heterocyclyl. In certain embodiments, R is optionally substituted C₁-C₂₀ alkyl. In certain embodiments, R is optionally substituted C₁-C₆ alkyl. In certain embodiments, R is optionally substituted C₃-C₈ cycloalkyl. In certain embodiments, R is optionally substituted aryl. In certain embodiments, R is optionally substituted heteroaryl. In certain embodiments, R is optionally substituted heterocyclyl.

As used herein, a disease or disorder is “alleviated” if the severity or frequency of at least one sign or symptom of the disease or disorder experienced by a patient is reduced.

As used herein, the term “analog” or “analogue” or “derivative” is meant to refer to a chemical compound or molecule made from a parent compound or molecule by one or more chemical reactions. As such, an analog can be a structure having a structure similar to that of the small molecule inhibitors described herein or can be based on a scaffold of a small molecule inhibitor described herein, but differing from it in respect to certain components or structural makeup, which may have a similar or opposite action metabolically. An analog or derivative of any of a small molecule inhibitor in accordance with the present disclosure can be used within the methods of the present disclosure.

As the term is used herein, “applicator” is used to identify any device including, but not limited to, a hypodermic syringe, pipette, nebulizer, vaporizer and the like, for administering the compounds and compositions used in the practice of the present disclosure.

As used herein, the term “container” includes any receptacle for holding the pharmaceutical composition. For example, in certain embodiments, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well-known in the art. It should be understood that the instructions for use of the pharmaceutical composition may be contained on the packaging containing the pharmaceutical composition, and as such the instructions form an increased functional relationship to the packaged product. However, it should be understood that the instructions can contain information pertaining to the compound’s ability to perform its intended function, e.g., treating, ameliorating, or preventing HSV-1 infection in a subject.

As used herein, a “disease” is a state of health of an animal wherein the animal cannot maintain homeostasis, and wherein if the disease is not ameliorated then the animal’s health continues to deteriorate.

As used herein, a “disorder” in an animal is a state of health in which the animal is able to maintain homeostasis, but in which the animal’s state of health is less favorable than it would be in the absence of the disorder. Left untreated, a disorder does not necessarily cause a further decrease in the animal’s state of health.

As used herein, the terms “effective amount” and “pharmaceutically effective amount” and “therapeutically effective amount” refer to an amount of an agent to provide the desired biological or therapeutic result. That result can be reduction and/or alleviation of the signs, symptoms, or causes of a disease or disorder, or any other desired alteration of a biological system. An appropriate effective amount in any individual case may be determined by one of ordinary skill in the art using routine experimentation.

As used herein, the term “endogenous” refers to any material from or produced inside an organism, cell, tissue or system.

As used herein, the term “exogenous” refers to any material introduced from or produced outside an organism, cell, tissue or system.

As used herein, the term “expression” is defined as the transcription and/or translation of a particular nucleotide sequence driven by its promoter.

As used herein, the term “HSV-1” refers to herpes simplex virus type 1.

As used herein, the terms “inhibit” and “inhibition” mean to reduce a molecule, a reaction, an interaction, a gene, an mRNA, and/or a protein’s expression, stability, function or activity by a measurable amount or to prevent entirely. “Inhibitors” are compounds that, e.g., bind to, partially or totally block stimulation, decrease, prevent, delay activation, inactivate, desensitize, or down regulate a protein, a gene, and an mRNA stability, expression, function and activity, e.g., antagonists.

“Instructional material,” as that term is used herein, includes a publication, a recording, a diagram, or any other medium of expression which can be used to communicate the usefulness of a composition of the present disclosure in a kit. The instructional material of the kit may, for example, be affixed to a container that contains a composition of the present disclosure or be shipped together with a container which contains a composition. Alternatively, the instructional material may be shipped separately from the container with the intention that the recipient uses the instructional material and a composition cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

As used herein, a “pharmaceutically acceptable carrier” means a pharmaceutically acceptable material, composition or carrier, such as a liquid or solid filler, diluent, excipient, solvent or encapsulating material, involved in carrying or transporting a compound(s) of the present disclosure within or to the subject such that it can perform its intended function. Typically, such compounds are carried or transported from one organ, or portion of the body, to another organ, or portion of the body. Each carrier must be “acceptable” in the sense of being compatible with the other ingredients of the formulation, and not injurious to the patient. Some examples of materials that can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer’s solution; ethyl alcohol; phosphate buffer solutions; and other non-toxic compatible substances employed in pharmaceutical formulations. As used herein “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds can also be incorporated into the compositions.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof.

As used herein, a viral strain is “resistant” to an antiviral agent if the minimum concentration necessary to inhibit the growth and/or kill the strain is higher than the average minimum concentration that inhibits the growth and/or kills other strains of the same virus. In certain embodiments, the minimum concentration of the antiviral agent necessary to inhibit the growth and/or kill the resistant strain is at least about 2 times higher, about 4 times higher, about 8 times higher, about 16 times higher, about 32 times higher, about 64 times higher, about 128 times higher, about 256 times higher, about 512 times higher, about 1,024 times higher, or about 2,048 times higher, about 10,000 times higher, or about 100,000 times higher than the average minimum concentration of the antiviral agent that inhibits the growth and/or kills other strains of the same virus.

By the term “specifically bind” or “specifically binds” as used herein is meant that a first molecule (e.g., an antibody) preferentially binds to a second molecule (e.g., a particular antigenic epitope), but does not necessarily bind only to that second molecule.

As used herein, the term “stapled” peptide refers to a peptide wherein the side chains of two or more amino acids are covalently linked through a linker. In certain embodiments, the term “stitched” peptide refers to a peptide wherein the side chains of three or more amino acids are covalently linked through a linker. As used herein, the term “stapled peptide” includes the term “stitched” peptide.

As used herein, the term “subject” or “patient” or “individual” includes humans and other animals, particularly mammals, and other organisms. Thus the methods are applicable to both human therapy and veterinary applications. In a specific embodiment, the patient is a mammal, and in certain embodiments the patient is human.

As used herein, the terms “treat,” “treating,” and “treatment,” refer to therapeutic or preventative measures described herein. The methods of “treatment” employ administration to a subject, in need of such treatment, a composition of the present disclosure, for example, a subject afflicted a disease or disorder, or a subject who is afflicted by any symptoms of the disease or disorder, in order to cure, delay, reduce the severity of, or ameliorate one or more symptoms of the disorder or recurring disorder, or in order to prolong the survival of a subject beyond that expected in the absence of such treatment.

Ranges: throughout this disclosure, various aspects of the present disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise. This applies regardless of the breadth of the range.

### Compounds and Compositions

The disclosure provides a compound comprising a stapled peptide of formula (I):
Xaa1-Xaa2-Xaa3-Xaa4-Xaa5-Xaa6-Xaa7-Xaa8-Xaa9-Xaa10-Xaa11-Xaa12-Xaa13-Xaa14 (I), or SEQ ID NO:1,
wherein the residues Xaa1-Xaa14 are defined as:
   Xaa3 is Arg or Lys;
   Xaa7 is His;
   Xaa11 is Asp;
   Xaa13 is Leu;
   Xaa14 is Ala;
   at least one residue pair selected from Xaa1-Xaa5, Xaa2-Xaa6, Xaa2-Xaa9, Xaa5-Xaa9, Xaa5-Xaa12, Xaa6-Xaa10, and Xaa8-Xaa12 is a residue pair which α-carbons are covalently linked through an independently selected linker, and
   the remaining residues selected from Xaa1, Xaa2, Xaa4, Xaa5, Xaa6, Xaa8, Xaa9, Xaa10, and Xaa12 are naturally occurring amino acids, wherein Xaa1 can be absent or Xaa1-Xaa2 can be absent;
   or a salt or solvate thereof.

In certain embodiments, if Xaa1 is absent, then the at least one residue pair is not Xaa2-Xaa6.

In certain embodiments, if Xaa1 is absent, then the N-terminus is not acylated (such as but not limited to, not formylated, acetylated, propionated, butyrated, and the like).

In certain embodiments, each linker is independently selected from:
—[(CH₂)₃—CH═CH—(CH₂)₃₋₆]—,
—[(CH₂)₈₋₁₁]—,
—[CH₂OCH₂—CH═CH—CH₂O(CH₂)₁₋₄]—,
—[CH₂O(CH₂)₄O(CH₂)₁₋₄]—,
—[(CH₂)(CH₂)ₘ₁—NH—C(═O)(CH₂)ₙ₁(CH₂)]—, wherein m1 and n1 are integers such that 3 ≤ (m1+n1) ≤ 6,

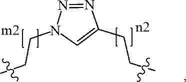

wherein m2 and n2 are integers such that 3 ≤ (m2+n2) ≤ 6,
—[(CH₂)(CH₂)ₘ₃—S—S—(CH₂)ₙ₃(CH₂)]—, wherein m3 and n3 are integers such that 0 ≤ (m3+n3) ≤ 2, and
—[(CH₂)(CH₂)ₘ₄S(CH₂)C(═O)NH(CH₂)ₙ₄(CH₂)]—, wherein m4 and n4 are integers such that 3 ≤ (m4+n4) ≤ 9.

In certain embodiments, the at least one residue pair is selected from Xaa1-Xaa5, Xaa2-Xaa6, Xaa5-Xaa9, Xaa6-Xaa10, and Xaa8-Xaa12, and the linker is selected from:
—[(CH₂)₃—CH═CH—(CH₂)₃]—,
—[(CH₂)₈]—,
—[CH₂OCH₂—CH═CH—CH₂O(CH₂)]—,
—[CH₂O(CH₂)₄O(CH₂)]—,
—[(CH₂)(CH₂)ₘ₁—NH—C(═O)(CH₂)ₙ₁(CH₂)]—, wherein m1 and n1 are integers such that (m1+n1) = 3,

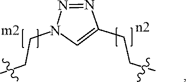

wherein m2 and n2 are integers such that (m2+n2) = 3,
—[(CH₂)(CH₂)ₘ₃—S—S—(CH₂)ₙ₃(CH₂)]—, wherein m3 and n3 are zero, and
—[(CH₂)(CH₂)ₘ₄S(CH₂)C(═O)NH(CH₂)ₙ₄(CH₂)]—, wherein m4 and n4 are integers such that 3 ≤ (m4+n4) ≤ 5.

In certain embodiments, the at least one residue pair is selected from Xaa2-Xaa9 and Xaa5-Xaa12, and the linker is selected from:
—[(CH₂)₃—CH═CH—(CH₂)₆]—,
—[(CH₂)₁₁]—,
—[CH₂OCH₂—CH═CH—CH₂O(CH₂)₄]—,
—[CH₂O(CH₂)₄O(CH₂)₄]—,
—[(CH₂)(CH₂)ₘ₁—NH—C(═O)(CH₂)ₙ₁(CH₂)]—, wherein m1 and n1 are integers such that (m1+n1) = 6,

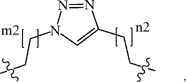

wherein m2 and n2 are integers such that (m2+n2) = 6,
—[(CH₂)(CH₂)ₘ₃—S—S—(CH₂)ₙ₃(CH₂)]—, wherein m3 and n3 are integers such that (m3+n3) = 2, and
—[(CH₂)(CH₂)ₘ₄S(CH₂)C(═O)NH(CH₂)ₙ₄(CH₂)]—, wherein m4 and n4 are integers such that 6 ≤ (m4+n4) ≤ 9.

In certain embodiments, Xaa1 is Glu, Val, Arg, or Ala.

In certain embodiments, Xaa2 is any naturally occurring amino acid, such as but not limited to Glu or Thr.

In certain embodiments, Xaa4 is any naturally occurring amino acid, such as but not limited to Arg or Ala.

In certain embodiments, Xaa5 is any naturally occurring amino acid, such as but not limited to Met.

In certain embodiments, Xaa6 is any naturally occurring amino acid, such as but not limited to Leu.

In certain embodiments, Xaa8 is any naturally occurring amino acid, such as but not limited to Arg.

In certain embodiments, Xaa9 is any naturally occurring amino acid, such as but not limited to Ala.

In certain embodiments, Xaa10 is any naturally occurring amino acid, such as but not limited to Phe.

In certain embodiments, Xaa12 is any naturally occurring amino acid, such as but not limited to Thr.

In certain embodiments, the compound is selected from the group consisting of:

Xaa1 Thr Arg Arg Xaa5 Leu His Arg Ala Phe Asp Thr Leu Ala (SEQ ID NO:2),
Glu Xaa2 Arg Arg Met Xaa6 His Arg Ala Phe Asp Thr Leu Ala (SEQ ID NO:3),
Glu Thr Arg Arg Met Leu His Xaa8 Ala Phe Asp Xaa12 Leu Ala (SEQ ID NO:4),
Xaa1 Thr Arg Arg Xaa5 Leu His Xaa8 Ala Phe Asp Xaa12 Leu Ala (SEQ ID NO:5),
Xaa1 Thr Arg Arg Xaa5 Leu His Arg Ala Phe Asp Xaa12 Leu Ala (SEQ ID NO:6),
Xaa1 Glu Arg Arg Xaa5 Leu His Arg Xaa9 Phe Asp Thr Leu Ala (SEQ ID NO:7),
Glu Thr Arg Arg Xaa5 Leu His Arg Ala Phe Asp Xaa12 Leu Ala (SEQ ID NO:8),
Glu Thr Arg Arg Xaa5 Leu His Arg Xaa9 Phe Asp Thr Leu Ala (SEQ ID NO:9),
Glu Thr Arg Arg Met Xaa6 His Arg Ala Xaa10 Asp Thr Leu Ala (SEQ ID NO:10),
Glu Xaa2 Arg Arg Met Leu His Arg Xaa9 Phe Asp Thr Leu Ala (SEQ ID NO:11),
Glu Xaa2 Arg Arg Met Xaa6 His Xaa8 Ala Phe Asp Xaa12 Leu Ala (SEQ ID NO:12),
Ala Glu Xaa1 Thr Arg Arg Xaa5 Leu His Arg Ala Phe Asp Thr Leu Ala (SEQ ID NO:13),
Arg Arg Xaa5 Leu His Arg Ala Phe Asp Xaa12 Leu Ala (SEQ ID NO:14),
Xaa2 Arg Arg Met Xaa6 His Arg Ala Phe Asp Thr Leu Ala (SEQ ID NO:15),
Val Thr Arg Arg Xaa5 Leu His Arg Xaa9 Phe Asp Thr Leu Ala (SEQ ID NO:16),
Val Val Thr Arg Arg Xaa5 Leu His Arg Xaa9 Phe Asp Thr Leu Ala (SEQ ID NO:17),
Arg Thr Arg Arg Xaa5 Leu His Arg Xaa9 Phe Asp Thr Leu Ala (SEQ ID NO:18),
Arg Arg Thr Arg Arg Xaa5 Leu His Arg Xaa9 Phe Asp Thr Leu Ala (SEQ ID NO:19),
Ala Thr Arg Ala Xaa5 Leu His Arg Xaa9 Phe Asp Thr Leu Ala (SEQ ID NO:20),
Ala Thr Lys Ala Xaa5 Leu His Arg Xaa9 Phe Asp Thr Leu Ala (SEQ ID NO:21),
Ala Thr Arg Ala Met Xaa6 His Arg Ala Xaa10 Asp Thr Leu Ala (SEQ ID NO:22),
Ala Gly Ala Thr Ala Glu Glu Thr Arg Arg Xaa5 Leu His Arg Xaa9 Phe
Asp Thr Leu Ala (SEQ ID NO:40),
Phe Gly Ala Val Gly Ala Gly Ala Thr Ala Glu Glu Thr Arg Arg Xaa5
Leu His Arg Xaa9 Phe Asp Thr Leu Ala (SEQ ID NO:41),
Lys Thr Arg Arg Xaa5 Leu His Arg Xaa9 Phe Asp Thr Leu Ala (SEQ ID NO:42),
Gln Thr Arg Arg Xaa5 Leu His Arg Xaa9 Phe Asp Thr Leu Ala (SEQ ID NO:43),
Asn Thr Arg Arg Xaa5 Leu His Arg Xaa9 Phe Asp Thr Leu Ala (SEQ ID NO:44),
Val Val Val Thr Arg Arg Xaa5 Leu His Arg Xaa9 Phe Asp Thr Leu Ala (SEQ ID NO:45),
Ile Thr Arg Arg Xaa5 Leu His Arg Xaa9 Phe Asp Thr Leu Ala (SEQ ID NO:46),
Leu Thr Arg Arg Xaa5 Leu His Arg Xaa9 Phe Asp Thr Leu Ala (SEQ ID NO:47),
Phe Thr Arg Arg Xaa5 Leu His Arg Xaa9 Phe Asp Thr Leu Ala (SEQ ID NO:48),
Trp Thr Arg Arg Xaa5 Leu His Arg Xaa9 Phe Asp Thr Leu Ala (SEQ ID NO:49),
Tyr Thr Arg Arg Xaa5 Leu His Arg Xaa9 Phe Asp Thr Leu Ala (SEQ ID NO:50).

In certain embodiments, the compound is selected from the group consisting of:

Xaa1 Thr Arg Arg Xaa5 Leu His Arg Ala Phe Asp Thr Leu Ala (SEQ ID NO:23),
Ala Glu Xaa3 Thr Arg Arg Xaa7 Leu His Arg Ala Phe Asp Thr Leu Ala (SEQ ID NO:26),
Arg Arg Xaa3 Leu His Arg Ala Phe Asp Xaa10 Leu Ala (SEQ ID NO:27),
Glu Thr Arg Arg Xaa5 Leu His Arg Xaa9 Phe Asp Thr Leu Ala (SEQ ID NO:30),
Val Thr Arg Arg Xaa5 Leu His Arg Xaa9 Phe Asp Thr Leu Ala (SEQ ID NO:31),
Val Val Thr Arg Arg Xaa6 Leu His Arg Xaa10 Phe Asp Thr Leu Ala (SEQ ID NO:32),
Arg Thr Arg Arg Xaa5 Leu His Arg Xaa9 Phe Asp Thr Leu Ala (SEQ ID NO:33),
Arg Arg Thr Arg Arg Xaa6 Leu His Arg Xaa10 Phe Asp Thr Leu Ala (SEQ ID NO:34),
Ala Thr Arg Ala Xaa5 Leu His Arg Xaa9 Phe Asp Thr Leu Ala (SEQ ID NO:35),
Ala Thr Lys Ala Xaa5 Leu His Arg Xaa9 Phe Asp Thr Leu Ala (SEQ ID NO:36),
Ala Thr Arg Ala Met Xaa6 His Arg Ala Xaa10 Asp Thr Leu Ala (SEQ ID NO:37),
Glu Thr Arg Arg Met Leu His Xaa8 Ala Phe Asp Xaa12 Leu Ala (SEQ ID NO:38),
Val Val Thr Arg Arg Cys Leu His Arg Cys Phe Asp Thr Leu Ala (SEQ ID NO:51).

In certain embodiments, at least one residue within the compound and/or peptide, and/or at the carboxy-terminus of the compound and/or peptide, and/or at the amino-terminus of the compound and/or peptide is methylated, amidated, acylated (such as, but not limited to, formylated, acetylated, propionated, butyrated, and the like), and/or substituted with any other chemical group without adversely affecting activity of the compound and/or peptide within the methods of the disclosure. In other embodiments, the N-terminus of the compound and/or peptide is acylated, such as but not limited to formylated, acetylated, propionated, butyrated, and the like. In other embodiments, the C-terminus of the compound and/or peptide is amidated. In certain embodiments, the N-terminus of the stapled peptide is linked via a peptidic bond to at least one additional amino acid residue. In certain embodiments, the at least one amino acid residue is a naturally occurring amino acid. In certain embodiments, the at least one additional amino acid residue is acylated (such as, for example, formylated, acetylated, propionated, butyrated, and the like) at its N-terminus.

The linker can be introduced in the compound and/or peptide using any techniques known in the art. Certain non-limiting types of linkers are discussed herein.

For example, a hydrocarbon linker can be introduced in the compound and/or peptide through metal-catalyzed olefin metathesis of a pair of amino acid residues comprising terminal alkenes. Similarly, an ether-containing linker can be introduced in the compound and/or peptide through metal-catalyzed olefin metathesis of a pair of amino acid residues comprising O-allyl ethers. In certain embodiments, the double bond resulting from the metathesis reaction can be hydrogenated to the corresponding single bond. See Ali, et al., 2019, Comput. Struct. Biotechnol. J. 17:263-281; Schafmeister, et al., 2000, J. Am. Chem. Soc. 122:5891-5892; Blackwell & Grubbs, 1998, Angew. Chem. Int. Ed. 37:3281-3284.

In certain embodiments, the amino acid residue is pentenyl glycine, or a derivative or enantiomer thereof, such as but not limited to (R)-2-amino-hept-6-enoic acid, (S)-2-amino-hept-6-enoic acid, (R)-2-methyl-2-amino-hept-6-enoic acid, or (S)-2-methyl-2-amino-hept-6-enoic acid.

In certain embodiments, the amino acid residue is O-allyl serine, or a derivative or enantiomer thereof, such as but not limited to (A)-O-allyl-serine, (S)- O-allyl-serine, (R)-2-methyl-O-allyl-serine, or (S)-2-methyl-O-allyl-serine.

In certain embodiments, the amino acid residue is octenyl glycine, or a derivative or enantiomer thereof, such as but not limited to (R)-2-amino-dec-9-enoic acid, (S)-2-amino-dec-9-enoic acid, (R)-2-methyl-2-amino-dec-9-enoic acid, and (S)-2-methyl-2-amino-dec-9-enoic acid.

In certain embodiments, the amino acid residue is 6-(allyloxy)-2-aminohexanoic acid, or a derivative or enantiomer thereof, such as but not limited to (R)-6-(allyloxy)-2-aminohexanoic acid, (S)-6-(allyloxy)-2-aminohexanoic acid, (R)-2-methyl-6-(allyloxy)-2-aminohexanoic acid, or (S)-2-methyl-6-(allyloxy)-2-aminohexanoic acid.

In certain embodiments, the amino acid residue is bis-pentenyl glycine, or a derivative or enantiomer thereof, such as but not limited to 2-amino-2-(pent-4-en-1-yl)hept-6-enoic acid. This amino acid residue can form a junction between two staples, leading to a stitched peptide.

In certain embodiments, stabilization of an α-helix can also be accomplished through side-chain intramolecular amide-bond formation between amine- and carboxy-side chain amino acids (Felix, et al., 1988, Intl. J. Pept. Protein Res. 32:441-454; Shepherd, et al., 2005, J. Am. Chem. Soc. 127:2974-2983). Residues that are useful for this approach include Asp, Glu, Orn, Lys, and any homologues thereof.

In certain embodiments, the amino acid residues in the at least one residue pair selected from Xaa1-Xaa5, Xaa2-Xaa6, Xaa2-Xaa9, Xaa5-Xaa9, Xaa5-Xaa12, Xaa6-Xaa10, and Xaa8-Xaa12 are selected from the group consisting of (S)-2-(4-pentenyl) alanine and (R)-2-(7-octenyl) alanine.

Cu (I)-catalyzed azide-alkyne cycloaddition (CuAAC) or the “Click” reaction is another mechanism of peptide stapling, and is also known as biocompatible ligation technique (Moses, et al., 2007, Chem. Soc. Rev. 36:1249-1262; Kawamoto, et al., 2012, J. Med. Chem. 55:1137-1146). In this case, an amino acid residue comprising a terminal alkyne group is reacted with an amino acid residue comprising an azido group in the presence of Cu(I) to form a 1,2,3-triazole.

Disulfide bridges between two thiol-containing residues can also be used as a stapling technique (Jackson, et al., 1991, J. Am. Chem. Soc. 113:9391-9392). In that particular case, the peptide is synthesized in its protected form (using for example, acetamidomethyl protective groups for the thiol groups); deprotection of the thiol groups and intramolecular oxidative coupling leads to the disulfide staple.

Further, stapling can be promoted by intramolecular reaction of a thiol group in an amino acid residue and an α-bromo amide group in another amino acid residue (Brunel & Dawson, 2005, Chem Commun. 2552-2554).

One skilled in the art would contemplate that stapling of residues within the peptides of the disclosure are not limited to the examples provided herein and encompass all forms of stapling known and recognized in the art.

Compounds of the disclosure may be prepared by the general schemes described herein, using the synthetic method known by those skilled in the art. The examples provided herein illustrate non-limiting embodiments of the disclosure. Variants of the polypeptides according to the present disclosure may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, and/or (iii) fragments of the polypeptides. Variants may be post-translationally, or chemically modified. Such variants are deemed to be within the scope of those skilled in the art from the teaching herein.

The compounds of the disclosure may possess one or more stereocenters, and each stereocenter may exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/ or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound of the disclosure, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compounds of the disclosure may exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compounds of the disclosure are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser’s Reagents for Organic Synthesis, Volumes 1-17 (John Wiley and Sons, 1991); Rodd’s Chemistry of Carbon Compounds, Volumes 1-5 and Supplementals (Elsevier Science Publishers, 1989); Organic Reactions, Volumes 1-40 (John Wiley and Sons, 1991), Larock’s Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000,2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

### Pharmaceutical Compositions

In one aspect, the present disclosure provides a pharmaceutical composition comprising the compound of the present disclosure and a pharmaceutically acceptable carrier.

In certain embodiments, the composition is formulated for topical administration.

In certain embodiments, the pharmaceutically acceptable carrier comprises liposomes. In certain embodiments, the liposomes are coated with chitosan.

In certain embodiments, the compound of the present disclosure is conjugated to a cyclic cell penetrating peptide. In certain embodiments, the cyclic cell penetrating peptide is CPP9. the cyclic cell penetrating peptide is conjugated to the compound of the present disclosure at the N-terminus or the C-terminus. The cyclic cell penetrating peptide is conjugated to the compound of the present disclosure via the linker.

### Methods

In one aspect, the present disclosure provides a method of treating, ameliorating, and/or preventing herpes keratitis in a subject. In another aspect, the present disclosure provides a method of inhibiting processive HSV-1 DNA synthesis in a subject. In another aspect, the present disclosure provides a method of treating, ameliorating, and/or inhibiting HSV-1 infection in a subject. In another aspect, the present disclosure provides a method of blocking and/or inhibiting HSV-1 propagation in a subject.

In certain embodiments, the method comprises administering to the subject an effective amount of a compound and/or composition of the disclosure. In other embodiments, the compound and/or composition of the disclosure is administered to the eye of the subject. In yet other embodiments, the compositions of the present disclosure comprise a pharmaceutically acceptable carrier.

In certain embodiments, the subject is further administered an anti-herpetic agent. In other embodiments, administration of the compound and/or composition reduces the effective amount of the anti-herpetic agent required to be administered to the subject to obtain the same therapeutic benefit. In yet other embodiments, the reduced effective amount of the anti-herpetic agent required to be administered to the subject to obtain the same therapeutic benefit results in a reduced frequency or severity of side effects experienced by the subject due to the anti-herpetic agent. In yet other embodiments, the anti-herpetic agent is at least one selected from the group consisting of acyclovir, famciclovir, ganciclovir, penciclovir, valacyclovir, vidarabine, and trifluridine.

### Administration/Dosage/Formulations

The regimen of administration may affect what constitutes an effective amount. The therapeutic formulations may be administered to the subject either prior to or after the onset of a disease or disorder contemplated herein. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient, preferably a mammal, more preferably a human, may be carried out using known procedures, at dosages and for periods of time effective to treat disease in the patient. An effective amount of the therapeutic compound necessary to achieve a therapeutic effect may vary according to factors such as the state of the disease or disorder in the patient; the age, sex, and weight of the patient; and the ability of the therapeutic compound to treat the disease or disorder contemplated herein in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the disclosure is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder contemplated herein in a patient.

The carrier may be a solvent or dispersion medium containing, for example, water, ethanol, polyol (for example, glycerol, propylene glycol, and liquid polyethylene glycol, and the like), suitable mixtures thereof, and vegetable oils.

In certain embodiments, the compositions of the disclosure are administered to the patient in dosages that range from one to five times per day or more. In other embodiments, the compositions of the disclosure are administered to the patient in range of dosages that include, but are not limited to, once every day, every two, days, every three days to once a week, once every two weeks, once every three weeks, once per month, once every 2 months, once every 3 months, and/or once every 1-12 weeks. It is readily apparent to one skilled in the art that the frequency of administration of the various combination compositions of the disclosure varies from individual to individual depending on many factors including, but not limited to, age, disease or disorder to be treated, gender, overall health, and other factors. Thus, the disclosure should not be construed to be limited to any particular dosage regime and the precise dosage and composition to be administered to any patient is determined by the attending physical taking all other factors about the patient into account.

Compounds of the disclosure for administration may be in the range of from about 1 µg to about 10,000 mg, about 20 µg to about 9,500 mg, about 40 µg to about 9,000 mg, about 75 µg to about 8,500 mg, about 150 µg to about 7,500 mg, about 200 µg to about 7,000 mg, about 350 µg to about 6,000 mg, about 500 µg to about 5,000 mg, about 750 µg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments thereinbetween.

In some embodiments, the dose of a compound of the disclosure is from about 1 mg and about 2,500 mg. In some embodiments, a dose of a compound of the disclosure used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the compounds of the disclosure can be administered ophthalmically, for example via intraocular or periocular contacting (with an eye drop or equivalent, in a non-limiting example) or injection. In other embodiments, the compounds are administered in a gel, a pegylated material, lipid nanoparticles, or liposomes. In other embodiments, the compounds themselves are pegylated or conjugated to a long-lasting biological molecule. In yet other embodiments, the compounds are formulated for slow delivery to the eye, for example using contact lenses comprising a polymer that releases the drug slowly, using punctual plugs, and/or using any delivery methodology that is known in the art and compatible with the present compounds.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of a disease or disorder in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., any analgesic agents.

Routes of administration of any of the compositions of the disclosure include oral, nasal, rectal, intravaginal, parenteral, buccal, sublingual or topical. The compounds for use in the disclosure may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastric, intrathecal, subcutaneous, intramuscular, intradermal, intra-arterial, intravenous, intrabronchial, inhalation, topical administration, and ophthalmic (including but not limited to topical, subconjunctival, subTenon’s, suprachoroidal, intravitreal, or subretinal).

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically acceptable excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

### Parenteral Administration

For parenteral administration, the compounds of the disclosure may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Ophthalmological Administrations

The disclosure contemplates administering to the eye the compounds useful within the disclosure. Any ophthalmological formulations can be useful within the present disclosure, as well as they allow for application of the compounds useful within the disclosure to the eye.

In a non-limiting example, the compositions of the disclosure comprise gamma cyclodextrin (or γ-cyclodextrin). A solution of gamma cyclodextrin can be prepared in water at concentrations up to its solubility limit of about 23.2 mg/mL. The pH of this cyclodextrin solution can then be adjusted to a pH at which the active compound is most soluble. The active compound is then added so that the molar ratio of gamma cyclodextrin to active compound is anywhere from about 1:1 to about 10:1. The resulting suspension or solution can then be stirred for a period of time (for example, 1 hour) after which the pH is adjusted to about 5-8, preferably about 6.5-7.5. The suspension or solution can be allowed to stir for up to about 24 hours after which it is used directly, diluted with buffer to a desired concentration, and/or lyophilized to provide a powder for reconstitution. The lyophilized powder can be suspended in an amount of water that will not dissolve the powder completely but will provide a fine suspension. This suspension can then be further formulated with a thickening agent to improve adherence to the eye. Thickening agents include, but are not limited to, carboxymethylcellulose (for example, at a concentration of about 0.05-5%), or other approved agents.

### Additional Administration Forms

Additional dosage forms of this disclosure include dosage forms as described in U.S. Pats. Nos. 6,340,475; 6,488,962; 6,451,808; 5,972,389; 5,582,837; and 5,007,790. Additional dosage forms of this disclosure also include dosage forms as described in U.S. Pat. Applications Nos. 20030147952; 20030104062; 20030104053; 20030044466; 20030039688; and 20020051820. Additional dosage forms of this disclosure also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the disclosure may be administered in the form of microparticles, for example, by injection or in the form of wafers or discs by implantation.

In one embodiment of the disclosure, the compounds of the disclosure are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present disclosure depends on the age, sex and weight of the patient, the current medical condition of the patient and the progression of the disease or disorder in the patient being treated. The skilled artisan is able to determine appropriate dosages depending on these and other factors.

A suitable dose of a compound of the present disclosure may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient’s status does improve, upon the doctor’s discretion the administration of the inhibitor of the disclosure is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient’s conditions has occurred, a maintenance dose is administered if necessary. Subsequently, the dosage or the frequency of administration, or both, is reduced, as a function of the viral load, to a level at which the improved disease is retained. In certain embodiments, patients require intermittent treatment on a long-term basis upon any recurrence of symptoms and/or infection.

The compounds for use in the method of the disclosure may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

### EXAMPLES

The disclosure is now described with reference to the following Examples. These Examples are provided for the purpose of illustration only, and the disclosure is not limited to these Examples, but rather encompasses all variations that are evident as a result of the teachings provided herein.

### Materials and Methods

### Cells

The African green monkey kidney epithelial cells (Vero and BSC-1) were cultured in Dulbecco’s modified Eagle’s medium (DMEM, Invitrogen) supplemented with 5% FBS, 2 mM _{L}-glutamine, 100 units/mL penicillin and 100 µg/mL streptomycin. Human primary corneal epithelial cells were purchased from ATCC® (PCS-700-010™). These human primary corneal epithelial cells were grown in serum-free corneal epithelial cell medium (ATCC® PCS-700-030™) supplemented with corneal epithelial cell growth kit (ATCC® PCS-700-040™) according to the manufacturer’s instruction.

### Plasmid Construction

Viral genomic DNA was isolated from HSV-1 strain KOS via treatment with Proteinase K at 56° C. for 1 h, followed by phenol-chloroform extraction and ethanol precipitation. The viral DNA was used to amplify full-length UL30 and UL42 genes by PCR with the following primer sets:

UL30-forward (5′- GACAAGCTTGCGATGTTTTCCGGTGGCGGC GGCCCGCT-3′) (SEQ ID NO:51), UL30-reverse (5′-GTGTCTAGATCATGCTAGAGTATCAAAGGCTCTATG-3′) (SEQ ID NO:52), UL42-forward (5′-GTCAAGCTTGGGATGACGGATTCCCCTGGCGGTGT-3′) (SEQ ID NO:53), and UL42-reverse (5′-GACTCTAGATCAGGGGAATCCAAAACCATACGGGGT-3′) (SEQ ID NO:54). Each of the forward primers contain a HindIII site and the original Kozak translation initiation sequences of the two genes, whereas the reverse primers comprise a XbaI site. PCR was conducted using Herculase enhanced DNA polymerase according to the manufacturer’s protocol (Agilent Technologies, Inc.). Amplified PCR products were ligated into the HindIII and XbaI site of PCDNA3.1 (+) plasmid (Invitrogen). Both cloned genes were confirmed by DNA sequencing. The vaccinia virus (VV) DNA polymerase E9, and Processivity factor A20 and D4 genes were cloned as previously described (J. Virol. 2010, 84:12325-12335). All constructs were used for in vitro translation using the TNT T7 coupled reticulocyte lysate system (Promega).

### Stapled Peptides

All peptides were synthesized by Bio-Synthesis, Inc. (Lewisville, TX) with reported purities >95%. In certain embodiments, stapled peptides were all hydrocarbon cross-linked at positions i, i+4, or i, i+7.

### In Vitro DNA Synthesis Assay

The assay was performed by the enzyme-linked immunosorbent assay (ELISA)-based Rapid Plate Assay (J. Virol. Methods 2000, 88:219-225; Methods Mol. Biol. 2005, 292:481-492; J. Med. Chem. 2008, 51:6563-6570), using in vitro translated viral DNA polymerase and processivity factor.

Briefly, the assay comprised a 100-nucleotide single-stranded DNA template that has biotin covalently linked to the 5′ end. The biotin-labeled oligonucleotide template was immobilized onto streptavidin, which was coated onto each well of a micro-titer plate. An oligonucleotide primer (15mer) was annealed to the 3′ end of the biotin-labeled DNA template. HSV-1 UL-30 and HSV-1 UL-42 (either in vitro translated or purified) were added and incubated with serial dilutions of the stapled peptides. DNA synthesis was measured by direct incorporation of the 4 dNTPs as well as the signal nucleotide, digoxigenin-dUTP (1:5, dig-dUTP:dTTP). A peroxidase-conjugated anti-digoxigenin antibody that recognizes the newly synthesized DNA generated a colorimetric reaction that was quantified with a plate reader at 405 nm.

### HSV-1 Infection of Human Primary Corneal Epithelial Cells

Human primary corneal epithelial cells were seeded in a 48-well plate (10⁴ cells/well) in 300 µL growth medium and cultured to 80-90% confluence. The human primary corneal epithelial cells were infected by absorbing HSV-1 (KOS) at ~ 100 PFU/well in 100 µL growth medium for 1 h. The cells were washed 4 times with PBS to remove unabsorbed virus, followed by treatment with S-pep7B at different dilutions in 300 µL growth medium containing 1% DMSO. At 72 h post-treatment, virus titers in the culture medium were determined by titration via standard plaque reduction assays in 48-well plates using Vero cells as described elsewhere herein.

### Inhibition of HSV-1 by Viral Plaque Reduction

Cells with ~90% confluence in 48-well plates were infected by absorbing virus (~100 PFU/well) for 1 h in 100 µL growth medium followed by adding 200 µL culture medium containing DMSO vehicle or serially diluted peptides or Acyclovir (Tocris Bioscience-Fisher Scientific) to each well of the plate. DMSO was maintained at 1% throughout the treatment. Cells were subsequently fixed and stained with 300 µL PBS containing 4% formaldehyde and 0.2% crystal violet overnight at room temperature. HSV-1 plaques were quantified at ~55 h post-infection of Vero cells. VV (WR strain) infection of BSC-1 cells was analyzed for plaque formation after 24 h. The plaque reduction assays were performed in duplicate and independently repeated twice. Cells were stained, and plaques counted under a dissecting microscope. Data were plotted on the GraphPad Prism.

### Stapled-Peptide Binding to UL42 Target Protein

The MST (Micro-Scale-Thermophoresis) binding assay was used. UL42 His-tagged protein was fluorescently labelled and incubated with serial dilutions of UL30 Stapled-Peptide. Samples were loaded onto capillaries and analyzed at 25° C. on a NanoTemper Monolith NT 115Pico instrument with a Pico-Red fluorescence channel. The data were fitted using the Hill model.

### Cytotoxicity and Cell Viability Assays

Cells were grown in 96-well plates to ~80% confluence and treated with peptides at different concentrations in 150 µL growth medium containing 1% DMSO. At 24 h post-treatment, 100 µL of the culture medium was used for the lactate dehydrogenase (LDH) cytotoxicity assay. In addition, the cells from the same treatment were assessed with an ATP-based cell viability assay. Briefly, after the growth medium was completely removed, the cells were lysed in 100 µL of 1% Triton X-100 in PBS per well at room temperature. After 10 min, 5 µL of lysate was used for ATP-based luciferase/luminescence assay according to the manufacturer’s protocol (Invitrogen, USA). The assays were performed in triplicate and independently repeated twice.

### Analysis of HSV-1 DNA Replication in Infected Cells Treated With S-Pep 7B Stapled Peptide

Confluent Vero cells in 48-well plates were infected by absorbing 10⁵ PFU of HSV-1 (MOI ~1) for 1 h in 100 µL of DMEM medium containing 5% FBS. Unabsorbed virus was removed by washing the wells 4 times with PBS. Cells were then treated with S-pep7B (SEQ ID NO:32) peptide in triplicate at different dilutions in 300 µL growth medium containing 1% DMSO. At 4 h post-treatment, cells of each triplicate treatment were combined and lysed with 20 mM Tris buffer (pH 7.5) containing 20 mM EDTA, 0.5% SDS, and 0.5 mg/mL proteinase K. Total DNA was then prepared from the lysed cells with phenol/chloroform extraction and ethanol precipitation, and used for UL42 gene amplification by PCR using the primers described above for plasmid construction. After agarose gel electrophoresis, UL42 DNA levels were quantitated using an image capture and analysis system (G:Box Chemi HR, Syngene) and normalized to that of the cellular house-keeping gene GAPDH (glyceraldehyde-3-phosphate dehydrogenase), which was amplified using primers (forward: 5′-ACATCATCCCTGCCTCTAC-3′ (SEQ ID NO:55), and reverse: 5′-TCAAAGGTGGAGGAGTGG-3′) (SEQ ID NO:56). The assays were independently repeated twice.

### Data Analysis

Half-maximal (EC₅₀, IC₅₀, and CC₅₀) values were obtained by non-linear regression fitting to a variable slope, four parameter dose-response model using the Prizm 6 software (GraphPad Software, La Jolla, CA).

### Example 1: Construction of Stapled Peptides That Mimic the Α-Helical Domain of the Extreme C-Terminus of UL30 DNA Polymerases

Stapled α-helical peptides have emerged as a new class of therapeutics applicable for targeting protein-protein interactions. Hydrocarbon stapling is a method of constraining an α-helical peptide by crosslinking two amino acid sidechains. Position of the staple on the α-helical peptide relies upon knowing the co-crystal structure of the protein from which it is derived in contact with its target protein. Moreover, unlike the typically disordered structure of unconstrained natural peptides, introduction of the staple as a brace to stabilize the helical structure decreases susceptibility to proteases by acting as a shield that can extend residence time. Hydrocarbon stapled peptides share key characteristics of both small molecules (e.g., cell permeability) and large biological drugs (e.g., highly specific target binding), and are increasingly being developed as therapeutics in many medical areas including cancer, metabolism, neuroscience, and infectious disease.

Described herein, in part, is the design of stapled peptides suitable to prevent the HSV-1 processivity factor UL42 from interacting with its cognate polymerase UL30 as a means of blocking viral DNA synthesis and infection.

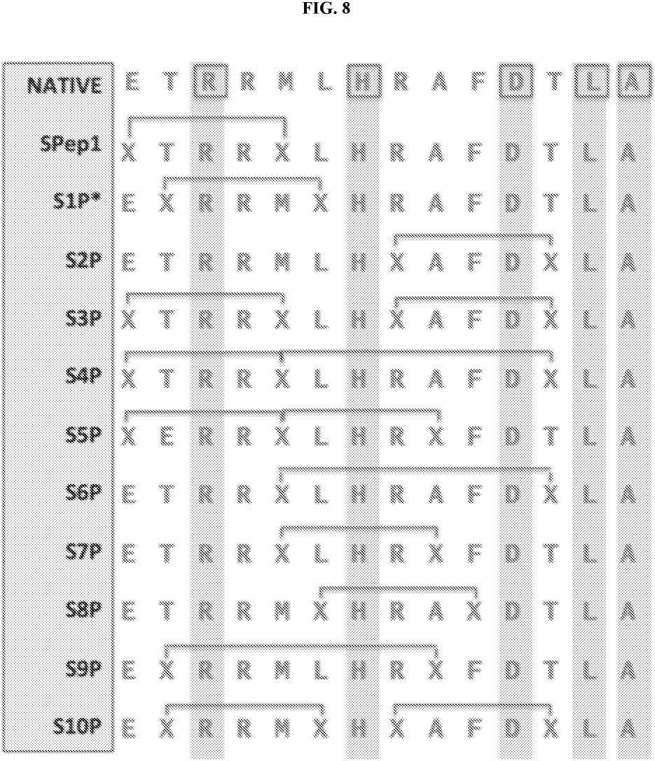

The co-crystal structure of UL42 and UL30 revealed that the C-terminal 36 amino acids of UL30 forms an αβα structure that binds to UL42. The extreme 15 amino acid C-terminal α-helix of UL30 is largely buried in a deep groove of UL42, with the interface contributed by electrostatic, hydrophobic, and hydrogen bonding interactions (FIG. 6). Based on this structure, a series of stapled peptides that mimic the extreme C-terminal α-helix were designed.

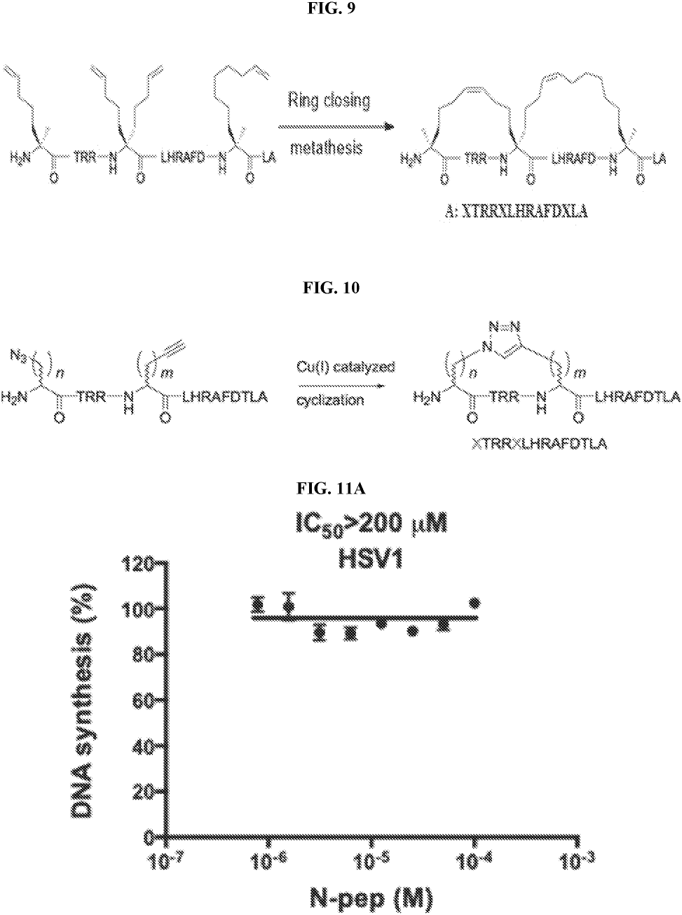

As shown in FIG. 7, peptide staples were introduced at positions i,i+4 or i,i+ 7 in order to stabilize the α-helix (3.6 amino acids/turn). Based on the UL30-UL42 co-crystal structure, the staples were positioned on the solvation-side of the α-helix of UL30 in order to prevent interfering with residues on the contact side required for making hydrogen bonds with UL42. Specifically, as shown in FIG. 6, these UL42 residues are D63, R64, and K289, each of which are engaged in hydrogen bonding interactions with UL30 residues R1224, H1228, D1232, L1234, and A1235 (depicted in orange and boxed).

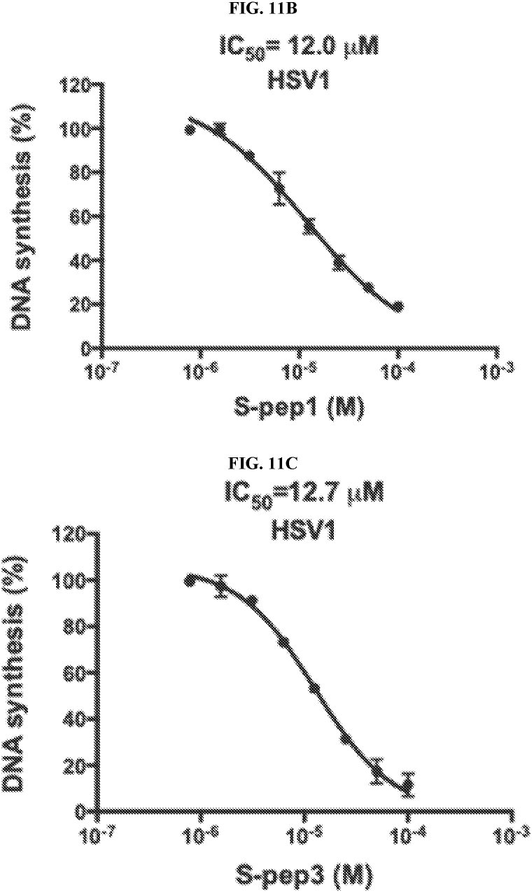

The design of hydrocarbon-stapled peptides is explored by varying the position and the length of the staple (FIG. 8). The i,i+4 staples can be accommodated beginning at T1223 (SlP*), R1229 (S2P), M1226 (S4P), and L1227 (S8P) and remain in the solvent exposed regions without interfering with binding. Similarly, i,i+7 staples can be accommodated beginning at M1226 (S6P) and T1223 (S9P). In certain embodiments, the incorporation of two staples at E1222+R1229 (S3P) or T1223+R1229 (S10P) further enhances helicity. In other embodiments, the pI for SPep-7 is optimized by incorporation of more acidic residues at permissive positions, such as the N-terminus thereby creating analogs of SPep-7B.

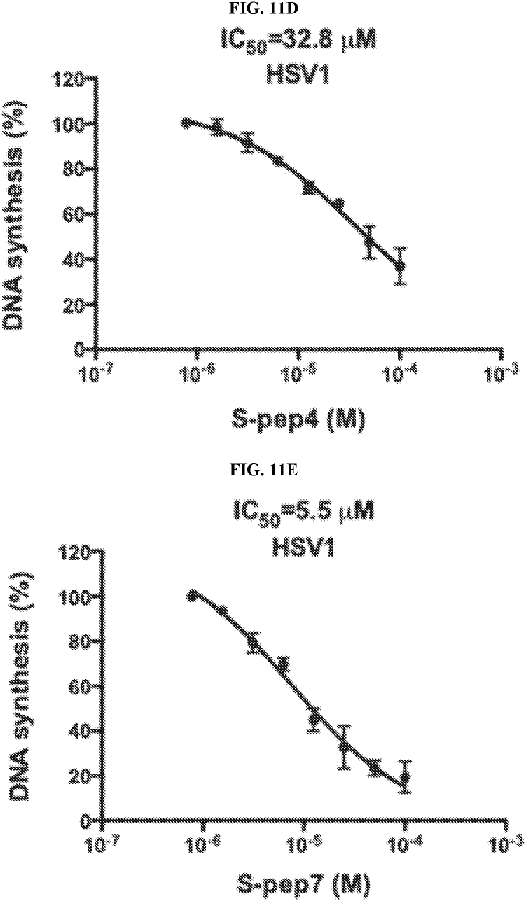

The peptides with the appropriate amino acid sequence, including the non-natural amino acids, can be prepared by routine resin-bound peptide synthesis, cyclized while on the resin, and then cleaved for purification. In certain embodiments, “stitched” peptides with two contiguous staples (such as i,i +4+7) have improved stability and enhanced cell penetrating ability relative to singly-stapled peptides. Three unnatural peptides containing four olefin groups are introduced, (S)-α-methyl,α-pentenylglycine at i, bis-pentenylglycine at i+4, and (5)-α-methyl,α-octenylglycine at i+7, for example, and then cyclized by ring closing metathesis. This technique can be applied to provide stitched peptides S5P and S7P (FIG. 9). Hydrogenation of the alkene in the staple retains helicity and may benefit binding. Therefore, hydrogenated versions of S1P through S10P, particularly any that have promising antiviral activity, can be prepared.

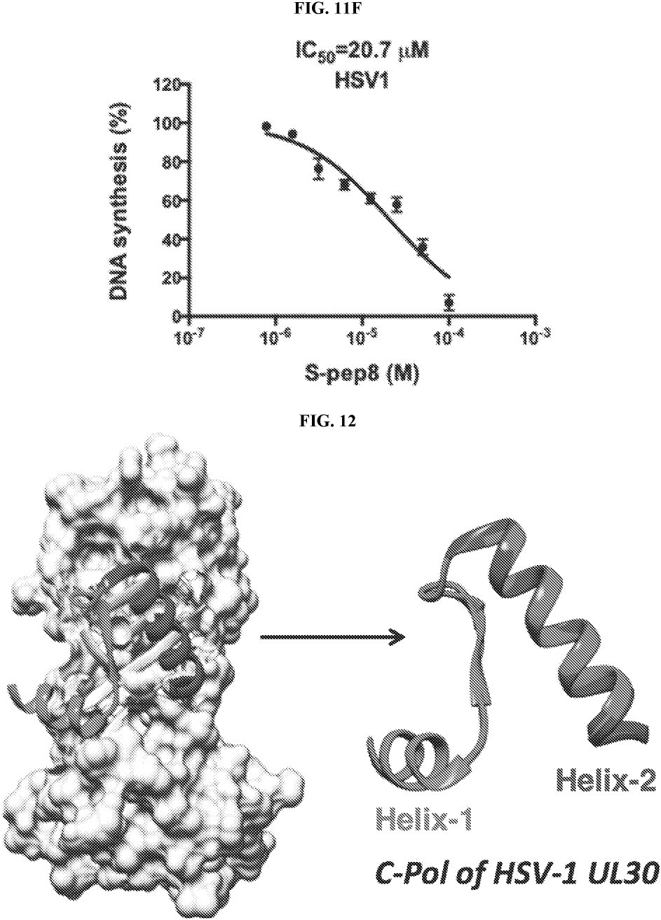

In addition to hydrocarbon links, another established method of peptide stapling is the use of alkyne-azide cyclization to form a triazole-containing linker. The necessary unnatural amino acids containing the azide and alkyne functional groups are commercially available with varying side chain lengths and with both absolute stereochemical configurations, allowing the easy exploration of a wide variety of stapled peptides. Because of their bioorthogonality, these amino acids can be incorporated into a peptide without the need for extra protecting groups. To synthesize triazole stapled peptides, azide containing amino acids is introduced in place of E1222 and alkyne containing amino acids at M1226 (FIG. 10). For i,i+4 stapling, staples where n+m = 5 or 6 are known to stabilize alpha-helical structures and the triazole can be accommodated in either orientation. The reverse orientation of the triazole is prepared by introducing the alkyne at E1222 and the azide at M1226. Use of both D- and L-amino acids allows evaluation of additional orientations of the staple. Similarly, for i,i+7 stapling, larger values of n and m are explored with the azide and alkyne incorporated at M1226 and T1233.

### Example 2: Stapled Peptides of UL30 Polymerase Block Processive DNA Synthesis in a Mechanistic Assay

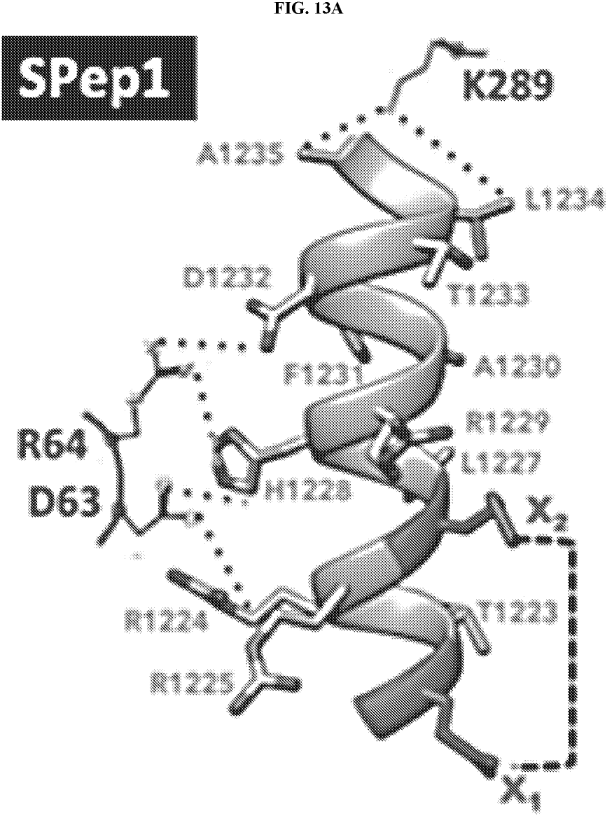

Using the immunosorbent assay (ELISA)-based Rapid Plate Assay described elsewhere herein, it was first tested if the UL30 stapled peptides could block in vitro processive DNA synthesis directed by recombinant UL30 and UL42 (FIGS. 11A-11F). As shown in FIG. 11A, the native control N-pep (SEQ ID NO:39) was totally incapable of inhibiting processive DNA synthesis in vitro (IC₅₀ > 200 µM).

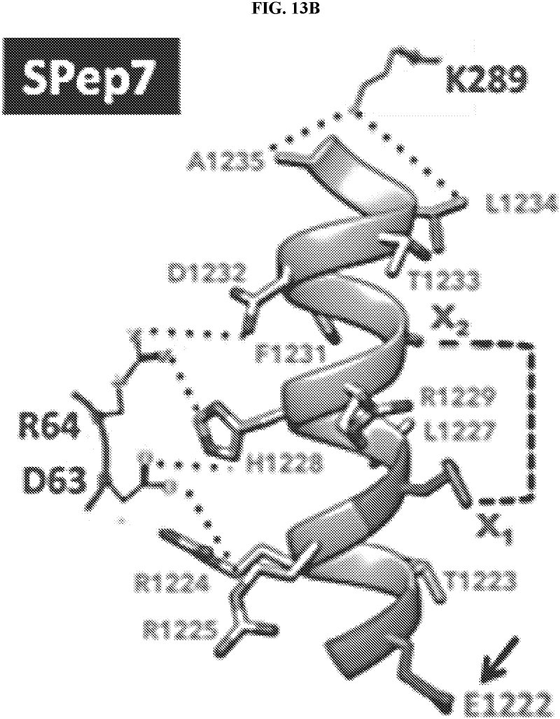

Based on the co-crystal structure (FIG. 12), amino acid peptides of C-Pol (UL30) with single- and double-staples located at different positions were designed (illustrative examples provided in FIG. 7 and FIG. 8). To explore stapled peptides capable of inhibiting processive DNA synthesis, three comparable constructs (S-pep1, S-pep2, and S-pep3) linking positions 1222 and 1226 were initially produced (FIG. 7). S-pep1 effectively inhibited processive DNA synthesis at IC₅₀ = 12.0 µM (FIG. 11B), suggesting the adoption of a stable helical structure capable of binding to UL42. A similar result was obtained for S-pep3 (IC₅₀ = 12.7 µM, FIG. 11C), which extends two additional amino acids (Ala¹²²⁰ and Glu¹²²¹) N-terminal of the staple. This result with S-pep3 indicated that the very N-terminus of the stapled peptide can be modified and still maintain its ability to contact the UL42 processivity factor and block DNA synthesis.

Elimination of the last four C-terminal amino acids (Asp¹²³²-Ala¹²³⁵) produced S-pep2, which lost activity (>200 µM), substantiating the importance of residues Leu¹²³⁴ and Ala¹²³⁵ that are known to make contact with Lys²⁸⁹ of UL42. In support of the requirement of Leu¹²³⁴ for peptide activity, the i,i+ 7 staple introduced at the corresponding Leu¹²²⁷ and Leu¹²³⁴ positions resulted in an inactive S-pep6 (>200 µM).

Accordingly, for each of the remaining peptides in this series (S-pep4, S-pep5, S-pep7, and S-pep8), the introduction of staples at residues known to contact the target protein UL42 was purposely avoided. As shown in FIGS. 11D-11F, S-pep4, S-pep7, and S-pep8 were able to inhibit processive DNA synthesis in vitro, with IC₅₀ = 32.8 µM, 5.5 µM, and 20.7 µM, respectively. S-pep5 was the only stapled peptide that failed to inhibit processive DNA synthesis, even though all residues known to contact the UL42 target remained intact. Without wishing to be bound by theory, comparison of S-pep5 with S-pep1, and S-pep3 suggests that in the context of this particular linking where position 1222 connects with 1226, Thr¹²²³ needs to be juxtaposed to residue Arg¹²²⁴, perhaps to maintain an essential bond angle for contacting Asp⁶³ of the UL42 target protein.

These data demonstrate that UL30 stapled peptides have a very high probability of functioning as inhibitors of processive DNA synthesis when residues that are required to contact the UL42 target protein are preserved. Conversely, there is a strong correlation between the inability of UL30 stapled peptides to block processive DNA synthesis and linkers which replace residues required to contact the UL42 target protein.

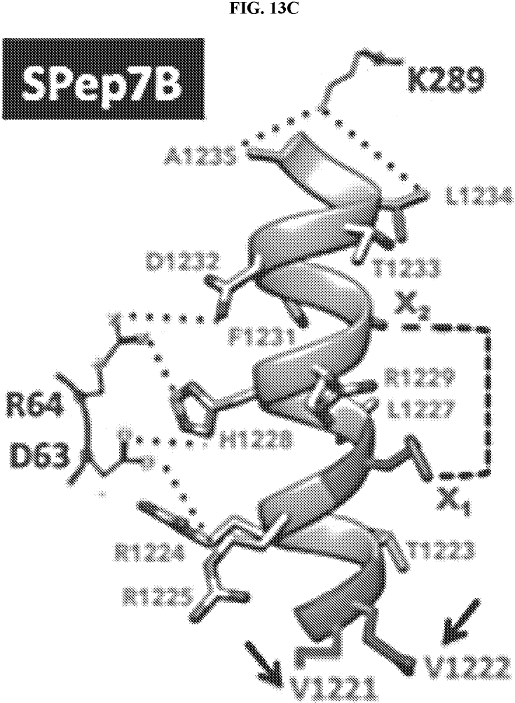

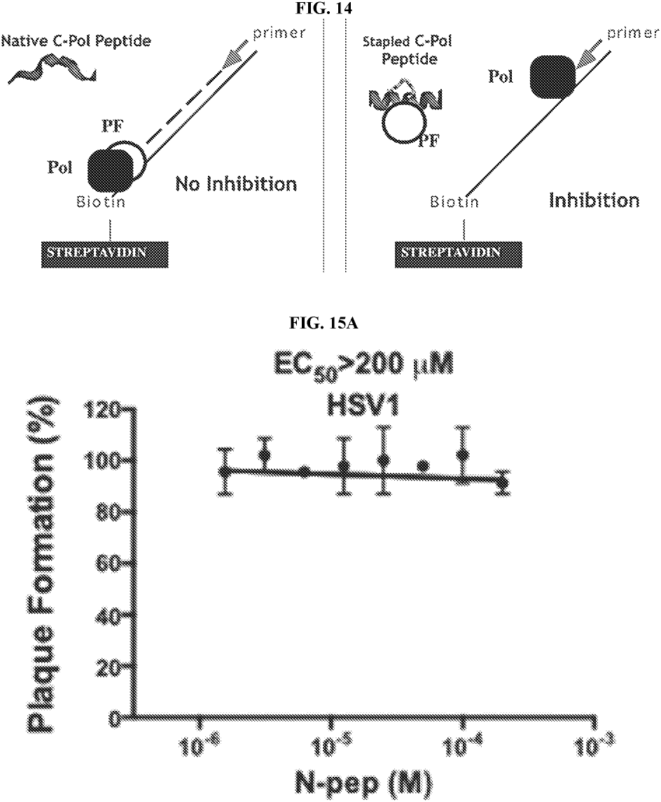

In certain non-limiting embodiments, the shown staples (or tethers), which provide rigidity, had linked-spacing of one full turn (i,i+4) or two full turns (i,i+7) of the helix to stay in helical register (3.6 amino acids/turn). In other embodiments, the staple was on the solvation side of the helix. Residues on the opposite side contact the PF target protein UL42. These UL42 residues are D63, R64 and K289 (FIGS. 13A-13C). Hydrogen bonds between residues of UL42 and C-Pol are shown as dotted lines. The IC₅₀ values recited herein were quantitated using the assay described in U.S. Pat. No. 6,204,028, which is incorporated herein in its entirety by reference (FIG. 14).

### Example 3: UL30 Polymerase Stapled Peptides Block HSV-1 Plaque Formation in Vero Cells

As demonstrated herein, unlike the stapled peptides of the disclosure, the non-stapled native peptide does not block DNA synthesis in vitro.

The plate assay described in U.S. Pat. 6,204,028 quantifies DNA synthesis by Pol/PF. FIG. 14 illustrates how native (unstapled) C-Pol peptide fails to inhibit HSV-1 processive DNA synthesis in vitro, whereas stapled SPep1 prevents PF (UL42) from binding to Pol and successfully inhibits HSV-1 DNA synthesis.

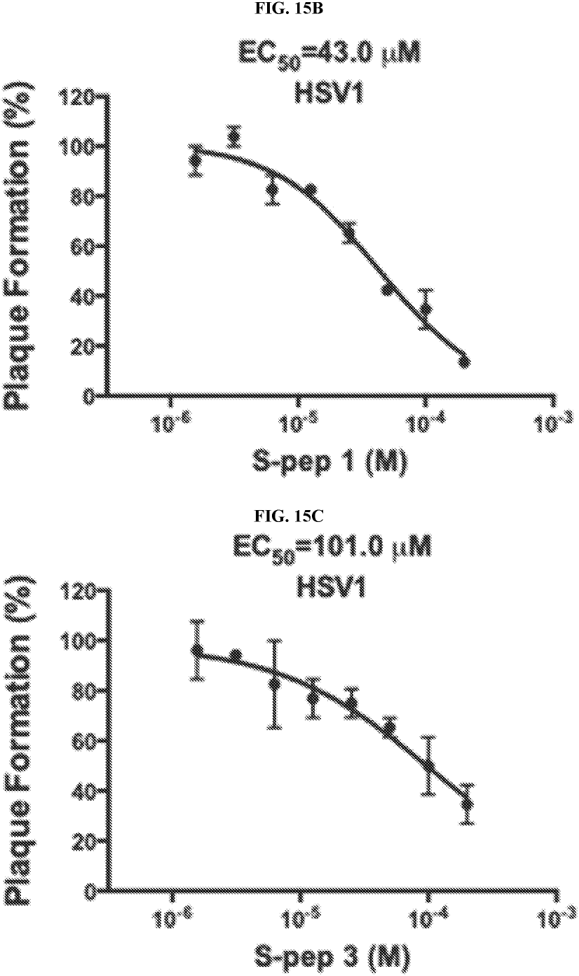

Standard viral plaque reduction assays were conducted in Vero cells to determine if any of the five stapled peptides (S-pep1/3/4/7/8) that were able to block processive DNA synthesis, could also block HSV-1 infection. To minimize possible interference of HSV-1 cell attachment and entry by the peptides, cells were first infected by absorbing the virus for 1 h, followed by treatment with peptides at serial dilutions. Consistent with the DNA synthesis observation, the native peptide N-pep showed no inhibition of HSV-1 infection (EC₅₀ > 200 µM, FIG. 15A). By contrast, inhibition was observed for S-pep1 (EC₅₀ = 43.0 µM) and S-pep7 (EC₅₀ 35.5 µM) (FIG. 15B and FIG. 15E). The EC₅₀ values of both S-pep3 and S-pep8 were approximately 100 µM (FIG. 15C and FIG. 15F). Additionally, S-pep4 was toxic at 12.5 µM, which precluded further optimization.

### Example 4: Properties of the Peptides of the Present Disclosure

Equilibrium solubility of peptides of the disclosure is tested by resuspending lyophilized peptide (1 mg) in PBS (1 ml) O/N, centrifuged and quantitated by LC/MSMS. For each peptide, quantitation is calculated as the average of 6 replicates. Alternatively, solubility studies are performed in DMSO (1%).

Size exclusion chromatography is used to resolve monomers from higher order aggregates. Lyophilized peptides are re-suspended in PBS (100-200 µM) and assayed by FPLC using a Superdex-75 (or Superdex-30 if greater resolution is required). MW standards include the native peptides and aprotinin (MW = 6500). If significant aggregation is observed (>10%), different buffering systems and salts, and excipients such as BSA, nonionic detergents or polymers (i.e. PEG300) at levels compatible with the bioassays can be evaluated.

One can correlate the degree of helicity imparted by the staples with protease resistance, target binding and ability to block infection. Without wishing to be limited by any theory, the staples on the peptides of the disclosure provide protease resistance. Briefly, peptides (25 to 50 µM) shown to be soluble and non-aggregating are dissolved in 5% acetonitrile/10 mM sodium phosphate, and Far-UV circular dichroism (CD) measurements are recorded in duplicate from 170-260 nm at 25° C. on an Aviv Model 410 spectrometer (Aviv Biomedical). CD signals are buffer-subtracted, converted to residual ellipticity (θ), and the helicity estimated by the variable selection (VARSLC) method. In certain embodiments, the increase in helicity over the native peptide is equal to or greater than 35%, 40%, 45%, 50%, 55%, 60%, 65%, 70%, 75%, 80%, 85%, 90%, 90%, 95%, or 100%.

Stapled peptides are often resistant to proteolysis because of stabilization of an alpha helical structure, which buries the amide backbone and blockade of the peptidase cleavage sites by the unnatural amino acids and the cross-linked staple. To investigate proteolytic susceptibilities, the proposed stapled peptides and their native counterparts are treated with trypsin and chymotrypsin according to the vendor’s (Sigma-Aldrich) published conditions. Stability of the parent peptide and appearance of proteolytic fragments over a 6 hour digestion period (triplicate samples/timepoint) is assayed by LC/MS. Target stability is >50% over 6 hrs. Reaction kinetics and rate constants are determined by plots of peptide concentration vs. time and linear regression analysis (Graphpad Prism). In the target 14 amino acid peptide sequence [^{N-1222}ETRRMLHRAFDTLA^{C-1235}] (SEQ ID NO:39), potential trypsin and chymotrypsin cleavage sites are located at positions [R1224, R1225, R1229] and [M1226, L1227, H1228, F1231, L1234], respectively (ExPASy Bioinformatics Resource Portal). Therefore, the two proteases interrogate stability of all contact residues in the peptides.

A stability screen was also performed with human plasma since many proteases (and protease inhibitors) found in tears are also present in plasma. Assays using heparinized mixed gender plasma measuring disappearance of parent peptide over a two hour period (triplicate samples/timepoint) are conducted. Target stability is >50% over 2 hrs. In certain embodiments, stability over 2 h assay is equal to or greater than 50%, 55%, 60%, 65%, 70%, 75%, 80%, 85%, 90%, 90%, 95%, or 99%.

Membrane disruptive activity is investigated in a hemolysis assay using freshly prepared human RBCs isolated from whole blood (BioIVT, Westbury NY) in PBS for 1 hr at 37° C. Released hemoglobin is measured by absorbance at 540 nm, and 1% Triton X-100 is the positive control, using Eq. 1:

$$\begin{matrix}\% \text{hemolysis} = \\ \left[\left({\text{A}}_{\text{540}} \text{sample} - {\text{A}}_{\text{540}} \text{blank}\right) / {\text{A}}_{540} \text{positive} \text{control}\right] \times 100\end{matrix}$$

Curve fitting by nonlinear regression (Prism Graphpad) is used to calculate a 50% hemolysis value (HC₅₀). Cellular uptake of the stapled peptides lacking hemolytic activity at 200 µM is evaluated relative to the native parent peptide using N-terminal beta-alanine fluorescein-5-isothiocyanate analogs (Bird, et al., 2002, Biopolymers 65(1): 10-20). The FITC tagged peptides are synthesized and incubated with human ocular epithelial cells in serum-free medium for up to 4 hours. Overall uptake is monitored by flow cytometry using propidium iodide as a control for membrane integrity. Confirmation of cellular uptake and subcellular localization is monitored by confocal fluorescence microscopy. In certain embodiments, sequence alterations (such as, but not limited to, glutamine substitution of the anionic glutamate residues where present) is employed.

Processivity of stapled peptides can be tested in a plate assay (U.S. Pat. No. 6,204,028). For target engagement, binding of stapled peptides is tested using SPR, ITC and MST biophysical assays (Kayris, et al., 2019, Expert Opin. Drug Discov. 1-14). Single amino acid substitutions are made on the three contact residues of UL42 (D63, R64, K289) and the mutated proteins are tested for binding to the stapled peptides to confirm specificity of engagement. UL30 is labelled in vitro and pull down-assays are performed with his-tagged UL42 in the presence of the stapled peptides to assay disruption of their binding interactions.

For infectivity standard plaque reduction assays are performed, and for toxicity both the ATP and cell proliferation assays (as described elsewhere herein) are performed.

Human organotypic cornea are used for testing the stapled α-helical peptides for herpes antiviral potency and toxicity. Briefly, human dermal fibroblasts are isolated from newborn foreskins, while corneal epithelial cells and keratocytes are isolated from corneo-scleral buttons obtained from local eye banks and sclerolimbal rings from corneal transplants. Since the mature organotypic corneal cultures are constructed from pooled corneo-scleral buttons, this ensures consistency between culture environments. After several weeks, each assembled culture has the morphology of 3D corneal epithelial tissue. Ten cultures are used for each stapled-peptide. Five concentrations of each peptide, as determined from the above studies, are used to obtain toxicity and antiviral activity curves. For antiviral activity, HSV-1 infection is performed according to methods known in the art (Drevets, et al., 2015, Graefes Arch Clin Exp Ophthalmol. 253(10): 1721-1728). Following viral adsorption at the air/liquid interface, followed by rinsing and change of medium, supernatants withdrawn from the organotypic cornea cultures are quantitated by plaque reduction. For toxicity, the MTT assay, which is a dye that is reduced to a purple metabolite only by mitochondria in metabolically active cells, can be used.

TABLE 1

Selected stapled peptides of the disclosure.

| | | |
|---|---|---|
| Name | Structure | IC₅₀, µM* (HSV1 UL30+UL42) |
| S-pep1 | Ac-XTRRXLHRAFDTLA (SEQ ID NO:23) | 12 |
| S-pep2 | Ac-XTRRXLBRAF-NH₂ (SEQ ID NO:25) | >200 |
| S-pep3 | Ac-AEXTRRXLHRAFDTLA (SEQ ID NO:26) | 12.7 |
| S-pep4 | Ac-RRXLHRAFDZLA (SEQ ID NO:27) | 32.8 |
| S-pep5 | Ac-XRRMXHRAFDTLA (SEQ ID NO:28) | >200 |
| S-pep6 | Ac-RRMXHRAFDTXA (SEQ ID NO:29) | >200 |
| S-pep7 | Ac-ETRRXLHRXFDTLA (SEQ ID NO:30) | 5.5 |
| S-pep7A | Ac-VTRRXLHRXFDTLA (SEQ ID NO:31) | 1.7 |
| S-pep7B | Ac-VVTRRXLHRXFDTLA (SEQ ID NO:32) | 1 |
| S-pep7C | Ac-RTRRXLHRXFDTLA (SEQ ID NO:33) | 1.7 |
| S-pep7D | Ac-RRTRRXLHRXFDTLA (SEQ ID NO:34) | 2 |
| S-pep7E | Ac-ATRAXLHRXFDTLA (SEQ ID NO:35) | |
| S-pep7F | Ac-ATKAXLHRXFDTLA (SEQ ID NO:36) | |
| S-pep7G | Ac-ATRAMXHRAXDTLA (SEQ ID NO:37) | |
| S-pep8 | Ac-ETRRMLHXAFDXLA (SEQ ID NO:38) | 20.7 |
| Non-stapled | Ac-ETRRMLHRAFDTLA (SEQ ID NO:39) | no inhibition |

* Note for stapling sites: peptides are either X-X or X-R8 stapled. X = (S)-2-(4-pentenyl) alanine; Z=(R)-2-(7-octenyl) alanine. IC₅₀ is a measure of HSV-1 DNA synthesis inhibition by the stapled-peptides. Sequence of each S-pep is derived from the C-terminus of Herpes Simplex Virus-1 (HSV-1) DNA Polymerase (UL30). S-peptides target the HSV-1 Processivity Factor UL42 which blocks binding of the native full-length UL30. As a result, HSV-1 DNA synthesis is inhibited. Ac = N-terminal acetyl group.

### Example 5: A Di-Valine Analog of S-Pep7 Blocks Processive DNA Synthesis More Effectively and Exhibits Increased Anti-Viral Potency Against HSV-1

It was investigated if S-pep7, selected from the primary pool of constructs described herein, could be further optimized. Results of the in vitro processive DNA synthesis assay (FIGS. 11A-11F) indicated that the extreme C-terminus of the stapled peptides comprising Leucine-Alanine cannot be altered or perturbed. This was made evident by comparing S-pep1 with S-pep2 and S-pep4 with S-pep6. By contrast, the extreme N-terminus was able to be modified without affected DNA synthesis, as revealed by comparing S-pep1 with S-pep3. Hence, being able to change the N-terminus of the stapled peptides without diminishing their abilities to block DNA synthesis, provided the rationale for testing amino acid substitutions that could potentially increase antiviral potency in HSV-1 infected cells.

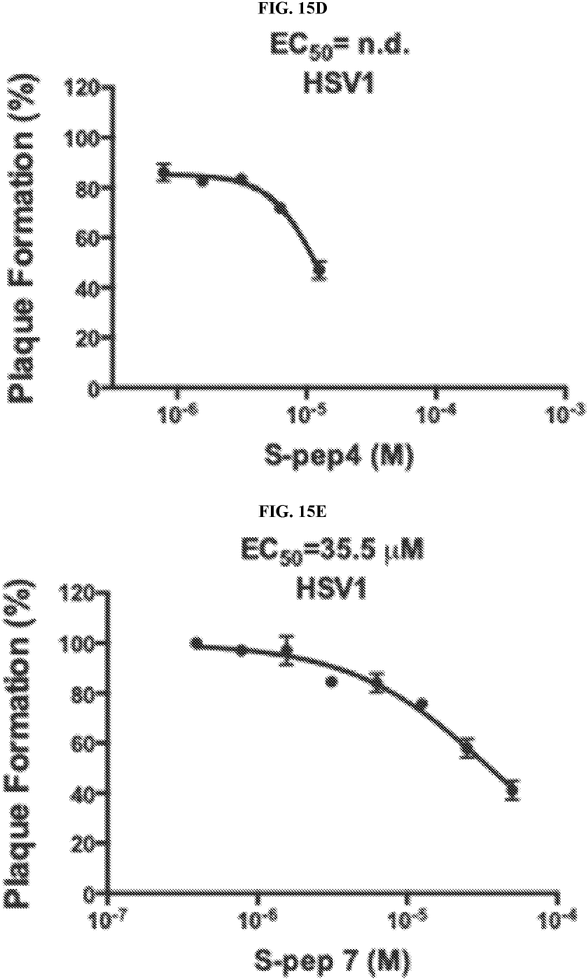

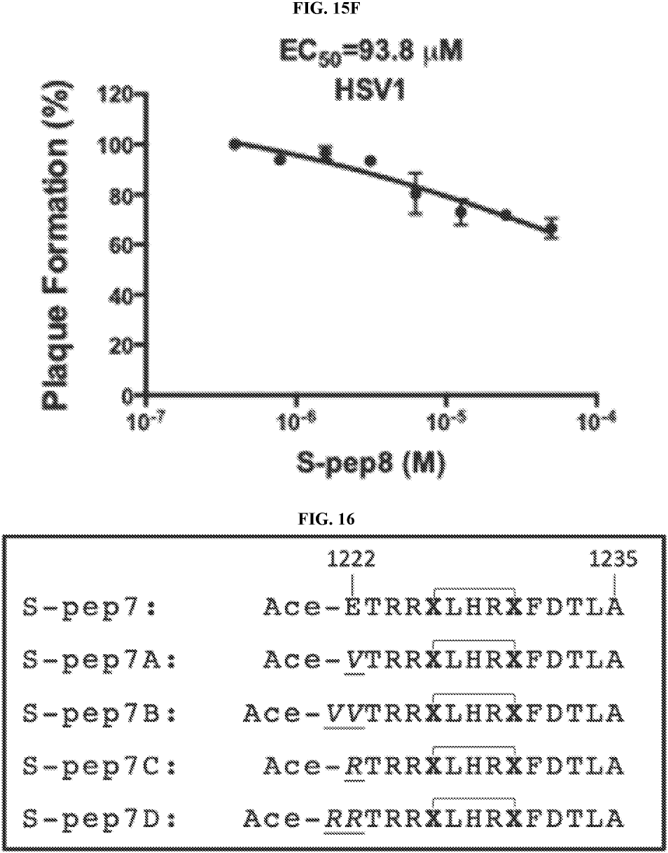

Since S-pep7 was the most effective stapled peptide capable of blocking both HSV-1 processive DNA synthesis (IC₅₀ = 5.5 µM) and cellular infection (EC₅₀ = 35.5 µM), alterations at its N-terminus which could enhance cellular uptake were incorporated. Predictions were made on the basis of biophysical features known to affect cellular uptake of stapled peptides including hydrophobicity, charge, amphipathicity, and structure. Negatively charged N-terminal residue Glu¹²²² was replaced with one and two hydrophobic Val residues to form S-pep7A and S-pep7B, respectively, or with one and two positively charged Arg residues to form S-pep7C and S-pep7D, respectively (FIG. 16). When compared to the parental S-pep7 (IC₅₀ = 5.5 µM), each of the N-terminal modified peptides was found to possess a comparable increase in ability to block HSV-1 processive DNA synthesis in vitro, with IC₅₀ values ranging between 1.0 µM (S-pep7B) to 2.0 µM (S-pep7D) (FIGS. 17A-17D, Table 1).

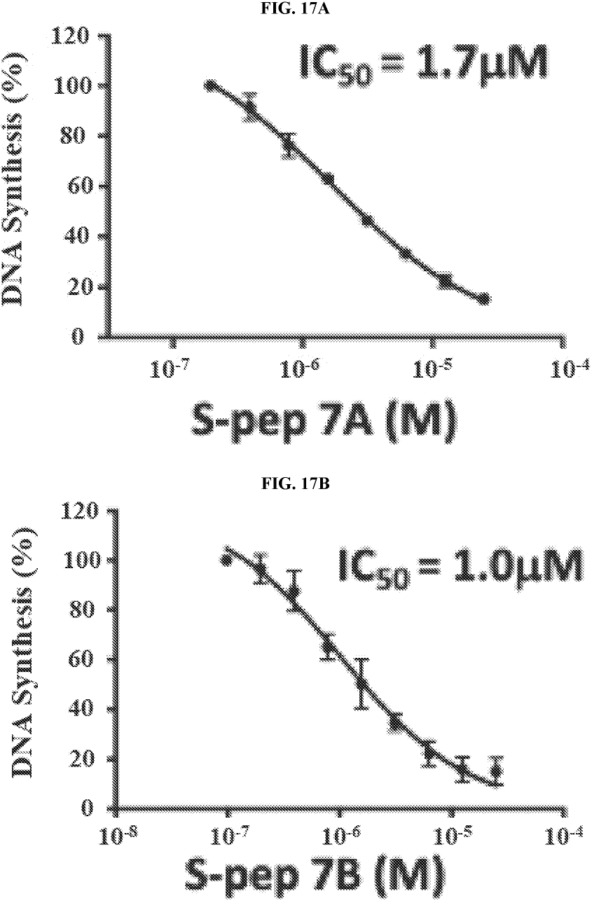

Next, it was determined whether the stapled peptides could also block HSV-1 from infecting Vero cells. Viral plaque reduction assays revealed that both the single and double Val substitutions had increased antiviral potency against HSV-1, with EC₅₀ = 9.6 µM and 9.8 µM for S-pep7A and S-pep7B, respectively (FIGS. 18A-18B) compared to parental S-pep7. As a direct comparison, Acyclovir was also tested for blocking HSV-1 plaque formation, which displayed an EC₅₀ = 0.81 µM (FIG. 18C). While S-pep7A and S-pep7B were less potent in blocking HSV-1 plaque formation in Vero cells, both peptides had IC₅₀ values around 1 µM in the in vitro processive DNA synthesis mechanistic assay. Formation of viral plaques could not be determined for S-pep7C and S-pep7D due to cellular toxicity.

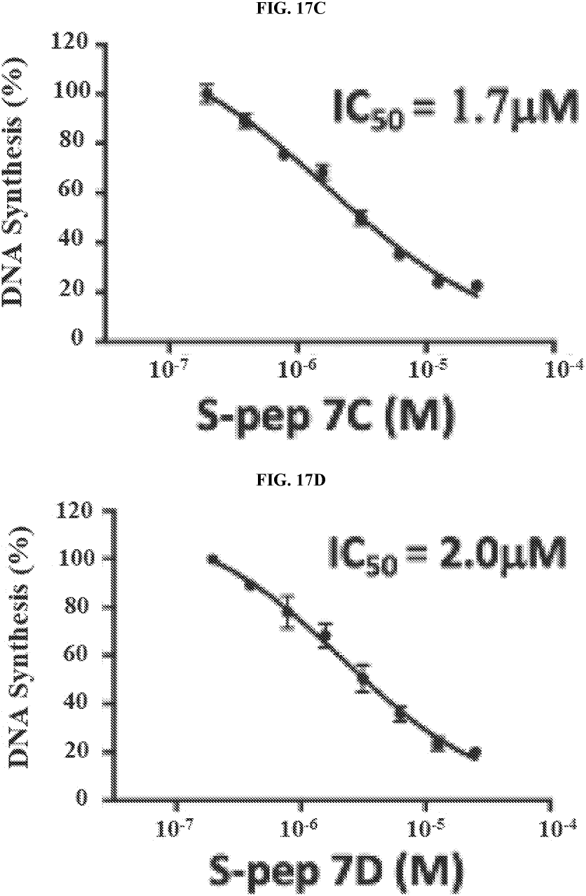

Cellular cytotoxicity of the N-terminal substituted stapled peptides was also assessed. S-pep7A caused observable cell death at 50 µM in the plaque assays. Both single and double Arg substitutions (S-pep7C and S-pep7D) exhibited dramatic cytotoxicity at 5 µM. By contrast, S-pep7B showed no visible cytotoxicity in the plaque reduction assays. Specifically, the measurements of intracellular ATP content and LDH leakage produced similar cytotoxicity information: CC₅₀ values of 114 µM for ATP and 126 µM for LDH (FIG. 19).

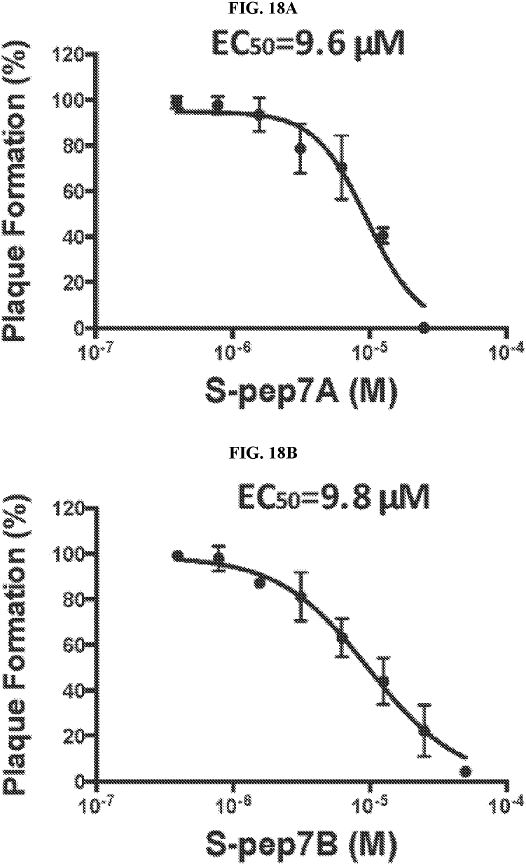

Next, the specificity of S-pep7B was evaluated by testing its antiviral activity directed against vaccinia virus (VV), which is completed unrelated to HSV-1. As shown in FIG. 20A S-pep7B completely failed to block in vitro DNA synthesis conducted by recombinantly expressed polymerase and processivity factor of VV (IC₅₀ > 50 µM). Correspondingly, S-pep7B did not inhibit VV from infecting cells (FIG. 20B), confirming its antiviral specificity as a herpes virus inhibitor.

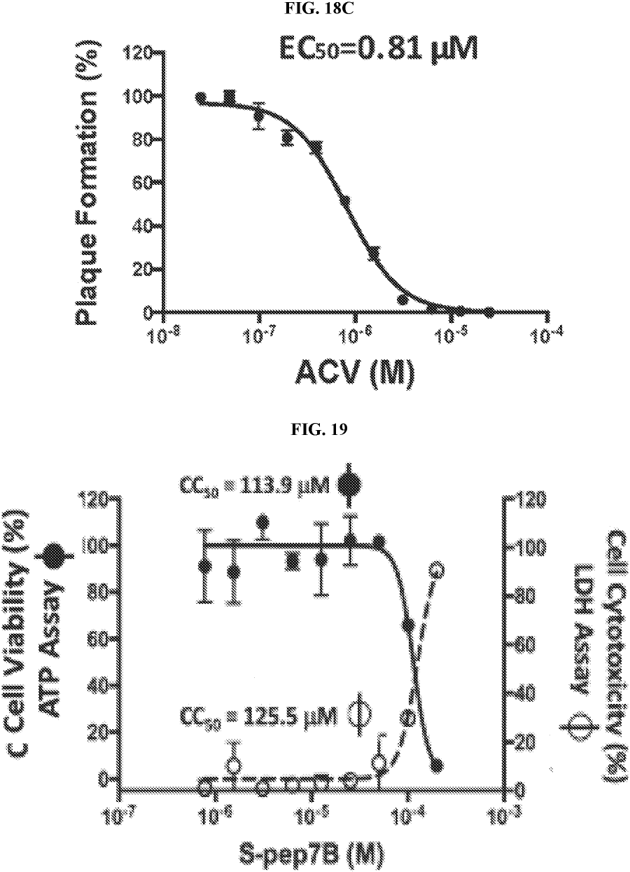

Experiments were subsequently performed to determine if the HSV-1 antiviral activity of S-pep7B was specifically due to the inhibition of viral DNA replication. Following 1 h absorption of HSV-1, cells were treated with vehicle or DMSO or increasing concentrations of S-pep7B. At 4 h post-treatment, viral genomic DNA was extracted from the cells and then used for amplification of the UL42 gene. As shown in FIG. 21, the UL42 DNA levels in infected cells were greatly reduced by S-pep7B in a dose dependent manner. At 50 µM, S-pep7B completely suppressed the viral DNA level to that of time point 0 h, which was the start of S-pep7B treatment (FIG. 21). These data clearly indicate that S-pep7B blocks HSV-1 infection by preventing viral DNA replication.

### Example 6: S-Pep7b Blocks HSV-1 Infection in Human Primary Corneal Epithelial Cells

Corneal infection by HSV-1 is the most frequent cause of visional loss by herpes keratitis, and as such, it was examined if S-pep7B could block HSV-1 infection in human primary corneal epithelial cells. Following 1 h absorption of HSV-1, the cells were treated with S-pep7B at increasing concentrations for 72 h. Unlike the clearly defined viral plaques formed on Vero cells following HSV-1 infection, distinctly quantifiable plaques were not apparent on human primary corneal epithelial cells due to morphological differences. To circumvent this issue, the amount of virus produced in the culture media of HSV-1 infected human primary corneal epithelial cells was collected and then quantitated by titration on Vero cells to evaluate plaque reduction by S-pep7B.

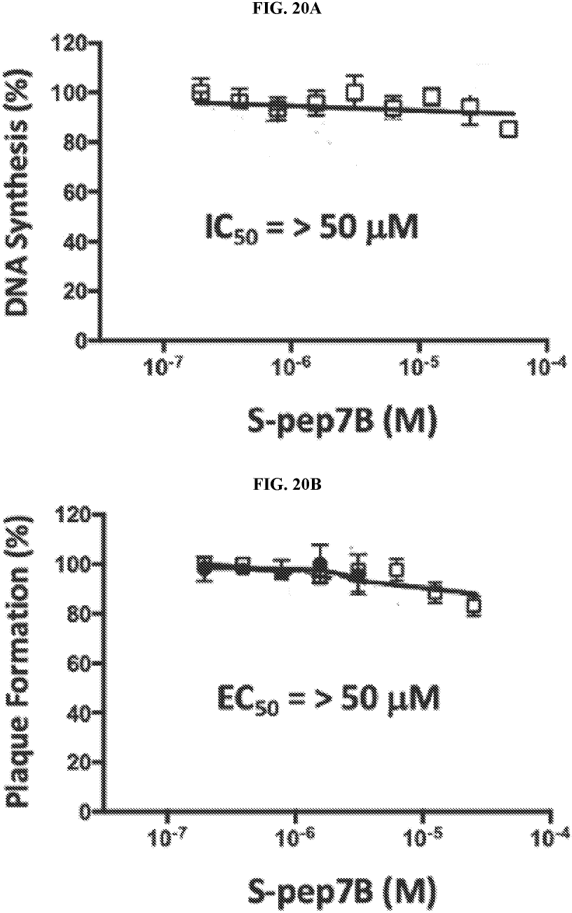

As shown in FIG. 22A, HSV-1 infection of human primary corneal epithelial cells was blocked by S-pep7B at an EC₅₀ = 2.7 µM. Furthermore, when the human primary corneal epithelial cells were tested for cytotoxicity in the presence of increasing concentrations of S-pep7B, a CC₅₀ = 31.2 µM was observed by ATP measurement (FIG. 22B). The determined Selectivity Index (SI) = 11.6 for S-pep7B in the human primary corneal epithelial cells proved to be identical to that observed in Vero cells. These results clearly demonstrate that S-pep7B is able to inhibit HSV-1 replication in human primary corneal epithelial cells.

### Example 7: S-Pep7b Blocks HSV-1 Infection in 3D Organotypic Corneal Cultures

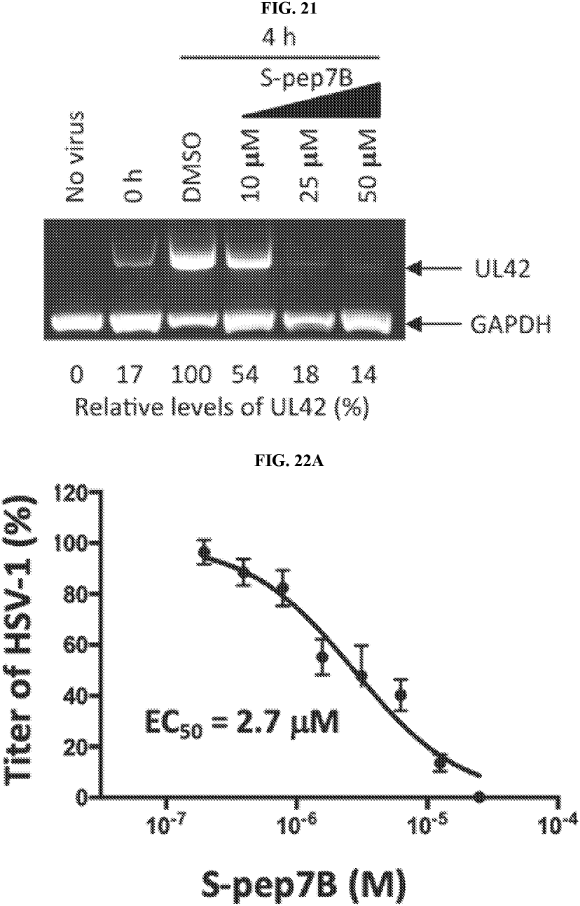

Cultures were assembled with human dermal fibroblasts isolated from newborn foreskins and primary human corneal epithelial cells isolated from unused sclerolimbal rings from corneal transplants. After three weeks, organotypic cultures with differentiated corneal epithelial cells overlaying a layer of fibroblasts are established, reproducing the organization of the anterior corneal surface (FIG. 23A).

The assembled 3D organotypic corneal culture was infected with HSV-1 by gently abrading the surface and topically applying 10⁶ PFU/mL of HSV-1. After 1 h, unabsorbed virus was removed, and the culture was incubated in medium containing 25 µM S-pep7B in 0.5% DMSO. Treatment occurred over a period of 5 days, in which the medium containing S-pep7B was refreshed every other day. The corneal tissues were examined microscopically each day. At the end of the treatment period, the 3D tissue was cut in half, using the first half to measure the virus titer by plaque assay and the other half for histological examination. Greater than 99% inhibition of HSV-1 titer by S-pep7B treated tissue was observed (FIG. 23B) and the complete absence of the HSV-1 structural gB protein was revealed (FIG. 23C), which is consistent with nearly total inhibition of viral infection. Significantly, nuclear staining with DAPI of this same 3D organotypic corneal tissue revealed an unremarkable nuclear morphology. Thus, the present results indicate that S-pep7B is not only efficacious in blocking herpes infection, but does not per se affect cellular morphology.

### Example 8: Enhanced Absorption of S-Pep7b Into Organotypic Corneal Cultures

Topical drug application is a direct means of treating herpes keratitis (HK), as the initial stages of HSV-1 infection are confined to the cornea, in which only six epithelial layers form the outer-most surface of the eye. While topical application may appear simple, it is in fact quite a challenge, owing to a number of physiological and anatomical barriers including secretion of lacrimal fluids, tears, and blinking, that dilute and wash away drugs and close-packed epithelial cells that form tight junctions that can prevent drug entry. In fact, in the absence of a transport vehicle, only 5% of a drug becomes absorbed through the corneal surface.

In certain embodiments, the peptide of the present disclosure is formulated with liposomes. Conventional liposomes have the benefit of enhancing permeation of poorly absorbed drugs to the eye, but have drawbacks of aggregation and leakage of the drug. Thus, in certain embodiments, the liposome is coated with chitosan, which prevents aggregation and increases encapsulation by preventing drug leakage, and elevates permeation by increasing muco-adhesion, resulting in greater retention that prolongs the rate of drug release.

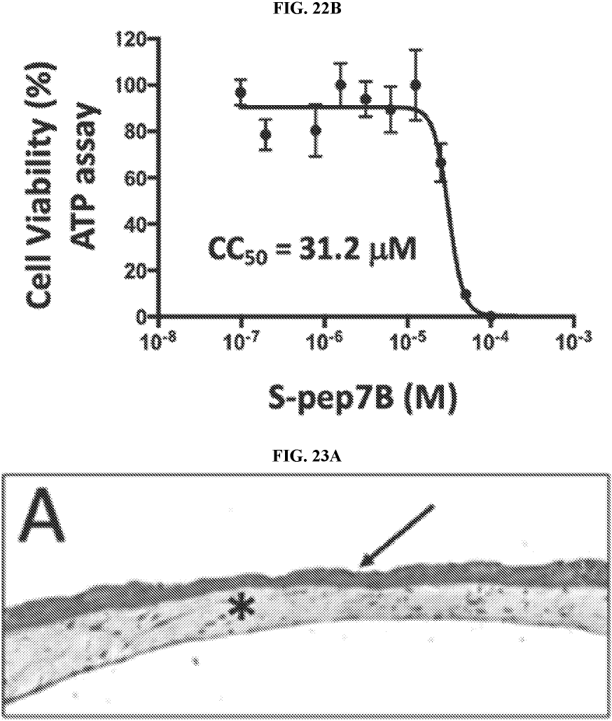

FIG. 24 illustrates a formulated chitosan coated liposome with S-pep7B in the aqueous core followed by penetration and release of the stapled peptide into the cytoplasm of the corneal epithelial cell by diffusion.

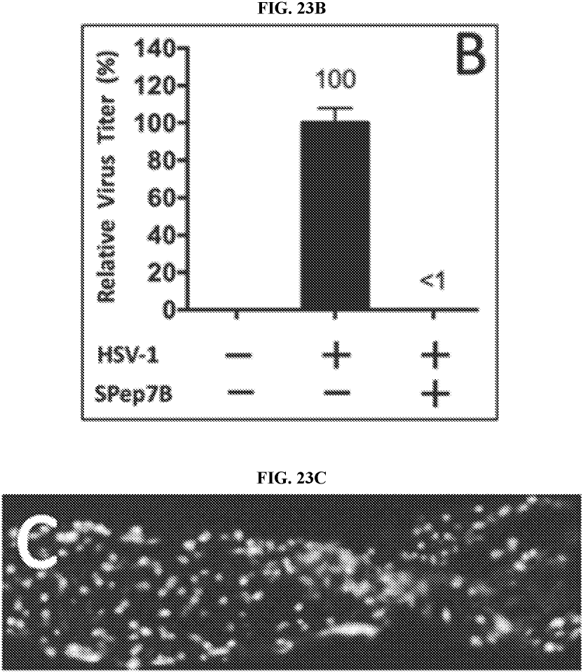

In other embodiments, S-pep7B is conjugated to cyclic peptides to enhance 3D absorption. In certain embodiments, the cyclic peptides are cell penetrating peptides (CPPs). CPPs are cyclic peptides which deliver molecules, which are otherwise membrane-impermeable, into a cell via endocytosis, and have been recently reported to specifically promote the uptake of stapled peptides. Non-limiting examples of CPPs include CPP9 (FIG. 25), an arginine-rich seven-membered cyclic peptide. In certain embodiments, the CPP can be attached to either the C-terminal or N-terminal end of a stapled peptide with a suitable linker to the carboxylic acid functionality on the glutamic acid residue of CPP9 in order to promote cell uptake at therapeutically relevant concentrations.

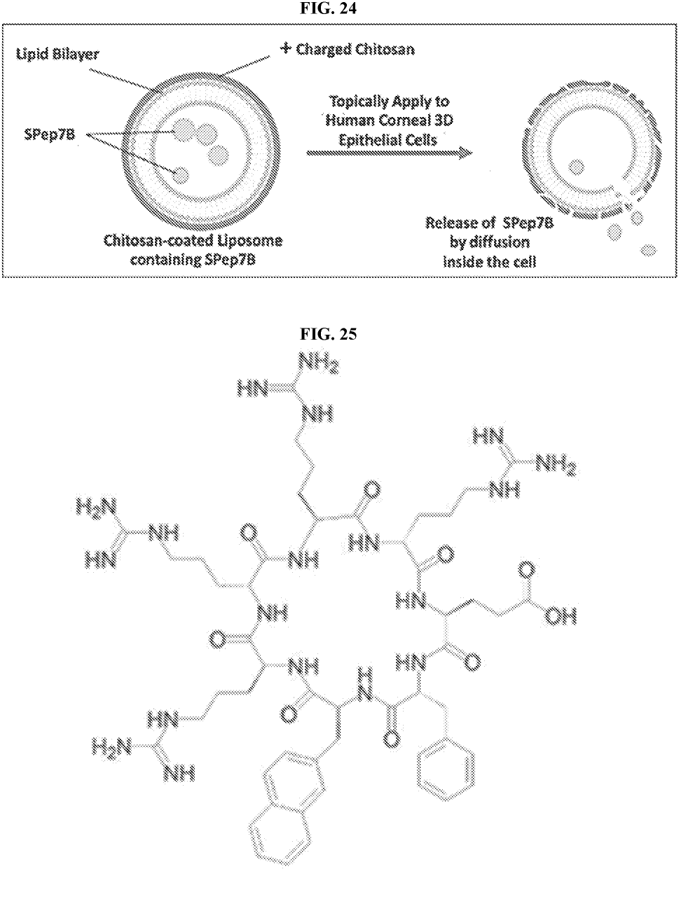

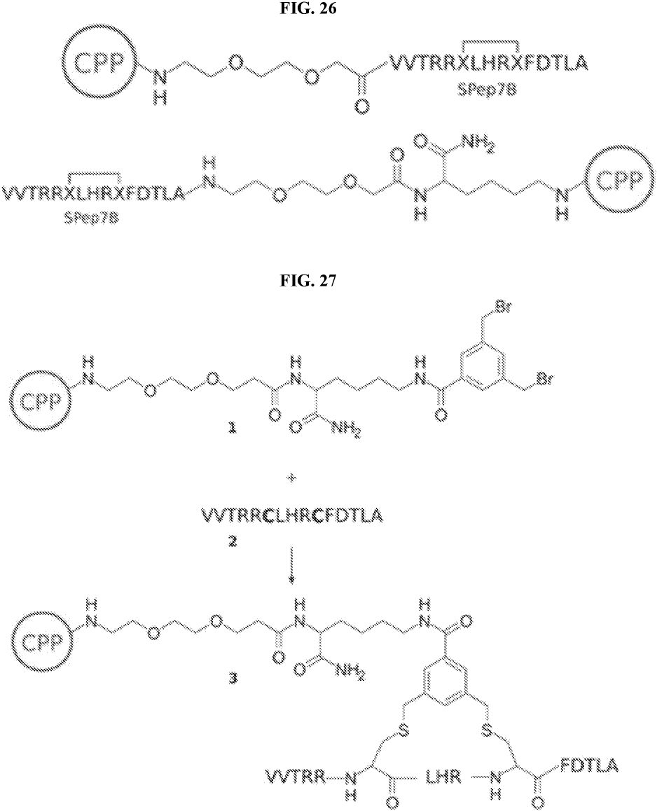

Suitable methods to prepare CPP-conjugated peptides of the present disclosure are described herein. In certain embodiments, CPP9 is conjugated to either end of S-pep7B (FIG. 26). In certain embodiments, CPP9 is conjugated to the staple itself. Using a cysteine based stapling strategy in place of the hydrocarbon-based staple present in S-pep7B allows for conjugation via the staple. For example, reaction of the peptide VVTRRCLHRCFDLTA (SEQ ID NO:59) in which the amino acid cystine is introduced at the two positions where the staple in S-pep7B is attached, with 1,3-bis(bromomethyl)benzene would provide a novel staple peptide with cystine cross-linking (FIG. 27).

### Example 9: N-Terminal Homologated Peptides

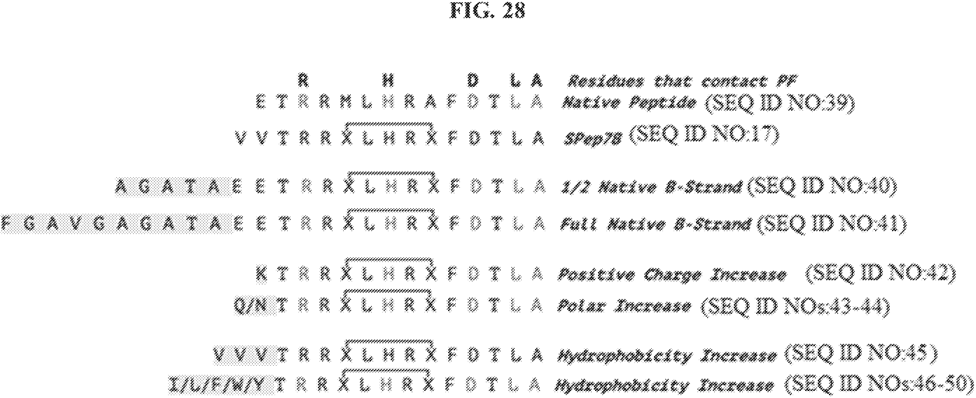

The N-terminus of the C-Pol stapled peptide has demonstrated greater tolerance to alteration than any other region, and thus represents a position for further optimization. However, negative charge at the N-terminus negates the ability to target the processive factor and block DNA synthesis. The natural N-terminal residues represent either one-half or the full β-strand that is juxtaposed to the α-helix of the native peptide. In certain embodiments, additional residues are added to the N-terminus of the natural peptide. In certain embodiments, 1-3 amino acid residues are added to the N-terminus of the natural peptide (FIG. 28). In certain embodiments, the amino acid residues added to the N-terminus of the natural peptide are positively charged. In certain embodiments, the amino acid residues added to the N-terminus of the natural peptide are hydrophobic. In certain embodiments, the N-terminus of the natural peptide is extended by three valine residues.

### Enumerated Embodiments

The following exemplary embodiments are provided, the numbering of which is not to be construed as designating levels of importance.

Embodiment 1 provides a compound comprising a stapled peptide of formula (I):
Xaa1-Xaa2-Xaa3-Xaa4-Xaa5-Xaa6-Xaa7-Xaa8-Xaa9-Xaa10-Xaa11-Xaa12-Xaa13-Xaa14 (I), SEQ ID NO:1,
wherein the residues Xaa1-Xaa14 are defined as:
   Xaa3 is Arg or Lys;
   Xaa7 is His;
   Xaa11 is Asp;
   Xaa13 is Leu;
   Xaa14 is Ala;
   at least one residue pair selected from Xaa1-Xaa5, Xaa2-Xaa6, Xaa2-Xaa9, Xaa5-Xaa9, Xaa5-Xaa12, Xaa6-Xaa10, and Xaa8-Xaa12 is a residue pair which α-carbons are covalently linked through an independently selected linker, and
   the remaining residues selected from Xaa1, Xaa2, Xaa4, Xaa5, Xaa6, Xaa8, Xaa9, Xaa10, and Xaa12 are naturally occurring amino acids, wherein Xaa1 can be absent or Xaa1-Xaa2 can be absent;
or a salt or solvate thereof

Embodiment 2 provides the compound of Embodiment 1, wherein each linker is independently selected from:
—[(CH₂)₃—CH═CH—(CH₂)₃₋₆]—,
—[(CH₂)₈₋₁₁]—,
—[CH₂OCH₂—CH═CH—CH₂O(CH₂)₁₋₄]—,
—[CH₂O(CH₂)₄O(CH₂)₁₋₄]—,
—[(CH₂)(CH₂)ₘ₁—NH—C(═O)(CH₂)ₙ₁(CH₂)]—, wherein m1 and n1 are integers such that 3 ≤ (m1+n1) ≤ 6,

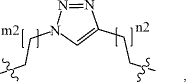

wherein m2 and n2 are integers such that 3 ≤ (m2+n2) ≤ 6,
—[(CH₂)(CH₂)ₘ₃—S—S—(CH₂)ₙ₃(CH₂)]—, wherein m3 and n3 are integers such that 0 ≤ (m3+n3) ≤ 2, and
—[(CH₂)(CH₂)ₘ₄S(CH₂)C(═O)NH(CH₂)ₙ₄(CH₂)]—, wherein m4 and n4 are integers such that 3 ≤ (m4+n4) ≤ 9.

Embodiment 3 provides the compound of any of Embodiments 1-2, wherein the at least one residue pair is selected from Xaa1-Xaa5, Xaa2-Xaa6, Xaa5-Xaa9, Xaa6-Xaa10, and Xaa8-Xaa12, and the linker is selected from:
—[(CH₂)₃—CH═CH—(CH₂)₃]—,
—[(CH₂)₈]—,
—[CH₂OCH₂—CH═CH—CH₂O(CH₂)]—,
—[CH₂O(CH₂)₄O(CH₂)]—,
—[(CH₂)(CH₂)ₘ₁—NH—C(═O)(CH₂)ₙ₁(CH₂)]—, wherein m1 and n1 are integers such that (m1+n1) = 3,

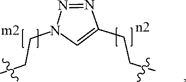

wherein m2 and n2 are integers such that (m2+n2) = 3,
—[(CH₂)(CH₂)ₘ₃—S—S—(CH₂)ₙ₃(CH₂)]—, wherein m3 and n3 are zero, and
—[(CH₂)(CH₂)ₘ₄S(CH₂)C(═O)NH(CH₂)ₙ₄(CH₂)]—, wherein m4 and n4 are integers such that 3 ≤ (m4+n4) ≤ 5.

Embodiment 4 provides the compound of any of Embodiments 1-3, wherein the at least one residue pair is selected from Xaa2-Xaa9 and Xaa5-Xaa12, and the linker is selected from:
—[(CH₂)₃—CH═CH—(CH₂)₆]—,
—[(CH₂)₁₁]—,
—[CH₂OCH₂—CH═CH—CH₂O(CH₂)₄]—,
—[CH₂O(CH₂)₄O(CH₂)₄]—,
—[(CH₂)(CH₂)ₘ₁—NH—C(═O)(CH₂)ₙ₁(CH₂)]—, wherein m1 and n1 are integers such that (m1+n1) = 6,

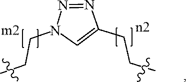

wherein m2 and n2 are integers such that (m2+n2) = 6,
—[(CH₂)(CH₂)ₘ₃—S—S—(CH₂)ₙ₃(CH₂)]—, wherein m3 and n3 are integers such that (m3+n3) = 2, and
—[(CH₂)(CH₂)ₘ₄S(CH₂)C(═O)NH(CH₂)ₙ₄(CH₂)]—, wherein m4 and n4 are integers such that 6 ≤ (m4+n4) ≤ 9.

Embodiment 5 provides the compound of any of Embodiments 1-4, wherein at least one applies:
(a) Xaa1 is Glu, Val, Arg, or Ala;
(b) Xaa2 is Glu or Thr;
(c) Xaa4 is Arg or Ala;
(d) Xaa5 is Met;
(e) Xaa6 is Leu;
(f) Xaa8 is Arg;
(g) Xaa9 is Ala;
(h) Xaa10 is Phe;
(i) Xaa12 is Thr.

Embodiment 6 provides the compound of any of Embodiments 1-5, wherein the compound consists essentially of the stapled peptide of formula (I).

Embodiment 7 provides the compound of any of Embodiments 1-5, wherein the compound consists of the stapled peptide of formula (I).

Embodiment 8 provides the compound of any of Embodiments 1-7, wherein at least one residue of the stapled peptide of formula (I) is methylated.

Embodiment 9 provides the compound of any of Embodiments 1-8, wherein the C-terminus of the stapled peptide of formula (I) is amidated.

Embodiment 10 provides the compound of any of Embodiments 1-9, wherein the N-terminus of the stapled peptide of formula (I) is acylated.

Embodiment 11 provides the compound of any of Embodiments 1-9, wherein the N-terminus of the stapled peptide linked via a peptidic bond to at least one additional amino acid residue.

Embodiment 12 provides the compound of Embodiment 11, wherein the at least one amino acid residue is a naturally occurring amino acid.

Embodiment 13 provides the compound of any of Embodiments 11-12, wherein the at least one additional amino acid residue acetylated at its N-terminus.

Embodiment 14 provides the compound of any of Embodiments 1-13, wherein if Xaa1 is absent, then the at least one residue pair is not Xaa2-Xaa6 or the N-terminus of the stapled peptide of formula (I) is not acylated.

Embodiment 15 provides the compound of any of Embodiments 1-14, wherein amino acid residues in the at least one residue pair selected from Xaa1-Xaa5, Xaa2-Xaa6, Xaa2-Xaa9, Xaa5-Xaa9, Xaa5-Xaa12, Xaa6-Xaa10, and Xaa8-Xaa12 are selected from the group consisting of (S)-2-(4-pentenyl) alanine and (R)-2-(7-octenyl) alanine.

Embodiment 16 provides the compound of any of Embodiments 1-15, which is selected from the group consisting of:

Xaa1 Thr Arg Arg Xaa5 Leu His Arg Ala Phe Asp Thr Leu Ala (SEQ ID NO:2),
Glu Xaa2 Arg Arg Met Xaa6 His Arg Ala Phe Asp Thr Leu Ala (SEQ ID NO:3),
Glu Thr Arg Arg Met Leu His Xaa8 Ala Phe Asp Xaa12 Leu Ala (SEQ ID NO:4),
Xaa1 Thr Arg Arg Xaa5 Leu His Xaa8 Ala Phe Asp Xaa12 Leu Ala (SEQ ID NO:5),
Xaa1 Thr Arg Arg Xaa5 Leu His Arg Ala Phe Asp Xaa12 Leu Ala (SEQ ID NO:6),
Xaa1 Glu Arg Arg Xaa5 Leu His Arg Xaa9 Phe Asp Thr Leu Ala (SEQ ID NO:7),
Glu Thr Arg Arg Xaa5 Leu His Arg Ala Phe Asp Xaa12 Leu Ala (SEQ ID NO:8),
Glu Thr Arg Arg Xaa5 Leu His Arg Xaa9 Phe Asp Thr Leu Ala (SEQ ID NO:9),
Glu Thr Arg Arg Met Xaa6 His Arg Ala Xaa10 Asp Thr Leu Ala (SEQ ID NO:10),
Glu Xaa2 Arg Arg Met Leu His Arg Xaa9 Phe Asp Thr Leu Ala (SEQ ID NO:11),
Glu Xaa2 Arg Arg Met Xaa6 His Xaa8 Ala Phe Asp Xaa12 Leu Ala (SEQ ID NO:12),
Ala Glu Xaa1 Thr Arg Arg Xaa5 Leu His Arg Ala Phe Asp Thr Leu Ala (SEQ ID NO:13),
Arg Arg Xaa5 Leu His Arg Ala Phe Asp Xaa12 Leu Ala (SEQ ID NO:14),
Xaa2 Arg Arg Met Xaa6 His Arg Ala Phe Asp Thr Leu Ala (SEQ ID NO:15),
Val Thr Arg Arg Xaa5 Leu His Arg Xaa9 Phe Asp Thr Leu Ala (SEQ ID NO:16),
Val Val Thr Arg Arg Xaa5 Leu His Arg Xaa9 Phe Asp Thr Leu Ala (SEQ ID NO:17),
Arg Thr Arg Arg Xaa5 Leu His Arg Xaa9 Phe Asp Thr Leu Ala (SEQ ID NO:18),
Arg Arg Thr Arg Arg Xaa5 Leu His Arg Xaa9 Phe Asp Thr Leu Ala (SEQ ID NO:19),
Ala Thr Arg Ala Xaa5 Leu His Arg Xaa9 Phe Asp Thr Leu Ala (SEQ ID NO:20),
Ala Thr Lys Ala Xaa5 Leu His Arg Xaa9 Phe Asp Thr Leu Ala (SEQ ID NO:21),
Ala Thr Arg Ala Met Xaa6 His Arg Ala Xaa10 Asp Thr Leu Ala (SEQ ID NO:22),
Ala Gly Ala Thr Ala Glu Glu Thr Arg Arg Xaa5 Leu His Arg Xaa9 Phe
Asp Thr Leu Ala (SEQ ID NO:40),
Phe Gly Ala Val Gly Ala Gly Ala Thr Ala Glu Glu Thr Arg Arg Xaa5
Leu His Arg Xaa9 Phe Asp Thr Leu Ala (SEQ ID NO:41),
Lys Thr Arg Arg Xaa5 Leu His Arg Xaa9 Phe Asp Thr Leu Ala (SEQ ID NO:42),
Gln Thr Arg Arg Xaa5 Leu His Arg Xaa9 Phe Asp Thr Leu Ala (SEQ ID NO:43),
Asn Thr Arg Arg Xaa5 Leu His Arg Xaa9 Phe Asp Thr Leu Ala (SEQ ID NO:44),
Val Val Val Thr Arg Arg Xaa5 Leu His Arg Xaa9 Phe Asp Thr Leu Ala (SEQ ID NO:45),
Ile Thr Arg Arg Xaa5 Leu His Arg Xaa9 Phe Asp Thr Leu Ala (SEQ ID NO:46),
Leu Thr Arg Arg Xaa5 Leu His Arg Xaa9 Phe Asp Thr Leu Ala (SEQ ID NO:47),
Phe Thr Arg Arg Xaa5 Leu His Arg Xaa9 Phe Asp Thr Leu Ala (SEQ ID NO:48),
Trp Thr Arg Arg Xaa5 Leu His Arg Xaa9 Phe Asp Thr Leu Ala (SEQ ID NO:49),
Tyr Thr Arg Arg Xaa5 Leu His Arg Xaa9 Phe Asp Thr Leu Ala (SEQ ID NO:50)

Embodiment 17 provides the compound of any of Embodiments 1-15, which is selected from the group consisting of:

Xaa1 Thr Arg Arg Xaa5 Leu His Arg Ala Phe Asp Thr Leu Ala (SEQ ID NO:23),
Ala Glu Xaa3 Thr Arg Arg Xaa7 Leu His Arg Ala Phe Asp Thr Leu Ala (SEQ ID NO:26),
Arg Arg Xaa3 Leu His Arg Ala Phe Asp Xaa10 Leu Ala (SEQ ID NO:27),
Glu Thr Arg Arg Xaa5 Leu His Arg Xaa9 Phe Asp Thr Leu Ala (SEQ ID NO:30),
Val Thr Arg Arg Xaa5 Leu His Arg Xaa9 Phe Asp Thr Leu Ala (SEQ ID NO:31),
Val Val Thr Arg Arg Xaa6 Leu His Arg Xaa10 Phe Asp Thr Leu Ala (SEQ ID NO:32),
Arg Thr Arg Arg Xaa5 Leu His Arg Xaa9 Phe Asp Thr Leu Ala (SEQ ID NO:33),
Arg Arg Thr Arg Arg Xaa6 Leu His Arg Xaa10 Phe Asp Thr Leu Ala (SEQ ID NO:34),
Ala Thr Arg Ala Xaa5 Leu His Arg Xaa9 Phe Asp Thr Leu Ala (SEQ ID NO:35),
Ala Thr Lys Ala Xaa5 Leu His Arg Xaa9 Phe Asp Thr Leu Ala (SEQ ID NO:36),
Ala Thr Arg Ala Met Xaa6 His Arg Ala Xaa10 Asp Thr Leu Ala (SEQ ID NO:37),
Glu Thr Arg Arg Met Leu His Xaa8 Ala Phe Asp Xaa12 Leu Ala (SEQ ID NO:38),
Val Val Thr Arg Arg Cys Leu His Arg Cys Phe Asp Thr Leu Ala (SEQ ID NO:51).

Embodiment 18 provides a method of treating or preventing herpes simplex virus-1′s (HSV-1′s) processive DNA synthesis in a subject infected with HSV-1, the method comprising administering to the subject a therapeutically effective amount of the compound of any of Embodiments 1-17.

Embodiment 19 provides a method of treating, ameliorating, and/or preventing HSV-1 propagation in a subject infected with HSV-1, the method comprising administering to the subject a therapeutically effective amount of the compound of any of Embodiments 1-17.

Embodiment 20 provides a method of treating, ameliorating, and/or preventing HSV-1 infection in a subject, the method comprising administering to the subject a therapeutically effective amount of the compound of any of Embodiments 1-17.

Embodiment 21 provides a method of treating and/or preventing herpes keratitis in a subject, the method comprising administering to the subject a therapeutically effective amount of the compound of any of Embodiments 1-17.

Embodiment 22 provides the method of any of Embodiments 18-21, wherein the compound is administered topically or ophthalmologically to the subject.

Embodiment 23 provides the method of any of Embodiments 18-22, wherein the compound is administered as part of a pharmaceutical composition.

Embodiment 24 provides the method of any of Embodiments 18-23, wherein the subject is further administered an anti-herpetic agent.

Embodiment 25 provides the method of Embodiment 24, wherein the anti-herpetic agent is at least one selected from the group consisting of acyclovir, famciclovir, ganciclovir, penciclovir, valacyclovir, vidarabine, and trifluridine.

Embodiment 26 provides the method of any of Embodiments 24-25, wherein the compound and the anti-herpetic agent are co-administered to the subject.

Embodiment 27 provides the method of any of Embodiments 24-26, wherein the compound and the anti-herpetic agent are co-formulated.

Embodiment 28 provides the method of any of Embodiments 18-27, wherein the subject is a mammal.

Embodiment 29 provides the method of Embodiment 28, wherein the mammal is a human.

Embodiment 30 provides a kit comprising the compound of any of Embodiments 1-17, the kit further comprising an applicator; and an instructional material for the use of the kit, wherein the instruction material comprises instructions for treating, ameliorating, and/or preventing herpes keratitis in a subject.

Embodiment 31 provides the kit of Embodiment 30, wherein the kit further comprises an anti-herpetic agent.

Embodiment 32 provides a pharmaceutical composition comprising the compound of any of Embodiments 1-17 and a pharmaceutically acceptable carrier.

Embodiment 33 provides the pharmaceutical composition of Embodiment 32, wherein the composition is formulated for topical administration.

Embodiment 34 provides the pharmaceutical composition of any of Embodiments 32-33, wherein the pharmaceutically acceptable carrier comprises liposomes.

Embodiment 35 provides the pharmaceutical composition of Embodiment 34, wherein the liposomes are coated with chitosan.

Embodiment 36 provides the pharmaceutical composition of any of Embodiments 32-35, wherein the compound of any of Embodiments 1-17 is conjugated to a cyclic cell penetrating peptide.

Embodiment 37 provides the pharmaceutical composition of Embodiment 36, wherein the cyclic cell penetrating peptide is CPP9.

Embodiment 38 provides the pharmaceutical composition of any of Embodiments 36-37, wherein the cyclic cell penetrating peptide is conjugated to the compound of any of Embodiments 1-17 at the N-terminus or the C-terminus.

Embodiment 39 provides the pharmaceutical composition of Embodiments 36-37, wherein the cyclic cell penetrating peptide is conjugated to the compound of any of Embodiments 1-17 via the linker.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the present disclosure. The appended claims are intended to be construed to include all such embodiments and equivalent variations.

1. A compound comprising a stapled peptide of formula (I):
Xaa1-Xaa2-Xaa3-Xaa4-Xaa5-Xaa6-Xaa7-Xaa8-Xaa9-Xaa10-Xaa11-Xaa12-Xaa13-Xaa14 (I), SEQ ID NO:1,
wherein the residues Xaa1-Xaa14 are defined as:
Xaa3 is Arg or Lys;
Xaa7 is His;
Xaa11 is Asp;
Xaa13 is Leu;
Xaa14 is Ala;
at least one residue pair selected from Xaa1-Xaa5, Xaa2-Xaa6, Xaa2-Xaa9, Xaa5-Xaa9, Xaa5-Xaa12, Xaa6-Xaa10, and Xaa8-Xaa12 is a residue pair which α-carbons are covalently linked through an independently selected linker, and
the remaining residues selected from Xaa1, Xaa2, Xaa4, Xaa5, Xaa6, Xaa8, Xaa9, Xaa10, and Xaa12 are naturally occurring amino acids, wherein Xaa1 can be absent or Xaa1-Xaa2 can be absent;
or a salt or solvate thereof.

2. The compound of claim 1, wherein each linker is independently selected from:
—[(CH₂)₃—CH═CH—(CH₂)₃₋₆]—,
—[(CH₂)₈₋₁₁]—,
—[CH₂OCH₂—CH═CH—CH₂O(CH₂)₁₋₄]—,
—[CH₂O(CH₂)₄O(CH₂)₁₋₄]—,
—[(CH₂)(CH₂)ₘ₁—NH—C(═O)(CH₂)ₙ₁(CH₂)]—,
wherein m1 and n1 are integers such that 3 ≤ (m1+n1) ≤ 6, 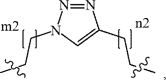
wherein m2 and n2 are integers such that 3 ≤ (m2+n2) ≤ 6,
—[(CH₂)(CH₂)ₘ₃—S—S—(CH₂)ₙ₃(CH₂)]—,
wherein m3 and n3 are integers such that 0 ≤ (m3+n3) ≤ 2, and
—[(CH₂)(CH₂)ₘ₄S(CH₂)C(═O)NH(CH₂)ₙ₄(CH₂)]—,
wherein m4 and n4 are integers such that 3 ≤ (m4+n4) ≤ 9.

3. The compound of claim 1, wherein one of the following applies:
(a) the at least one residue pair is selected from Xaa1-Xaa5, Xaa2-Xaa6, Xaa5-Xaa9, Xaa6-Xaa10, and Xaa8-Xaa12, and the linker is selected from:
—[(CH₂)₃—CH═CH—(CH₂)₃]—,
—[(CH₂)₈]—,
—[CH₂OCH₂—CH═CH—CH₂O(CH₂)]—,
—[CH₂O(CH₂)₄O(CH₂)]—,
—[(CH₂)(CH₂)ₘ₁—NH—C(═O)(CH2)ₙ₁(CH₂)]—,
wherein m1 and n1 are integers such that (m1+n1) = 3, 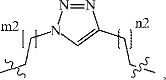
wherein m2 and n2 are integers such that (m2+n2) = 3,
—[(CH₂)(CH₂)ₘ₃—S—S—(CH₂)ₙ₃(CH₂)]—, wherein m3 and n3 are zero, and
—[(CH₂)(CH₂)ₘ₄S(CH₂)C(═O)NH(CH₂)n4(CH₂)]—,
wherein m4 and n4 are integers such that 3 ≤ (m4+n4) ≤ 5; and
(b) the at least one residue pair is selected from Xaa2-Xaa9 and Xaa5-Xaa12, and the linker is selected from:
—[(CH₂)₃—CH═CH—(CH₂)₆]—,
—[(CH₂)₁₁]₋,
—[CH₂OCH₂—CH═CH—CH₂O(CH₂)₄]—,
—[CH₂O(CH₂)₄O(CH₂)₄]—,
—[(CH₂)(CH₂)ₘ₁—NH—C(═O)(CH₂)ₙ₁(CH₂)]—,
wherein m1 and n1 are integers such that (m1+n1) = 6, 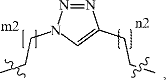
wherein m2 and n2 are integers such that (m2+n2) = 6,
—[(CH₂)(CH₂)ₘ₃—S—S—(CH₂)ₙ₃(CH₂)]—,
wherein m3 and n3 are integers such that (m3+n3) = 2, and
—[(CH₂)(CH₂)ₘ₄S(CH₂)C(═O)NH(CH₂)ₙ₄(CH₂)]—,
wherein m4 and n4 are integers such that 6 ≤ (m4+n4) ≤ 9.

4. (canceled)

5. The compound of claim 1, wherein at least one applies:
(a) Xaa1 is Glu, Val, Arg, or Ala;
(b) Xaa2 is Glu or Thr;
(c) Xaa4 is Arg or Ala;
(d) Xaa5 is Met;
(e) Xaa6 is Leu;
(f) Xaa8 is Arg;
(g) Xaa9 is Ala;
(h) Xaa10 is Phe;
(i) Xaa12 is Thr.

6. The compound of claim 1, wherein the compound consists essentially of the stapled peptide of formula (I).

7. The compound of claim 1, wherein the compound consists of the stapled peptide of formula (I).

8. The compound of claim 1, wherein at least one of the following applies:
(a) at least one residue of the stapled peptide of formula (I) is methylated;
(b) the C-terminus of the stapled peptide of formula (I) is amidated; and
(c) the N-terminus of the stapled peptide linked via a peptidic bond to at least one additional amino acid residue, optionally wherein the at least one amino acid residue is a naturally occurring amino acid, and optionally wherein the at least one additional amino acid residue is acetylated at its N-terminus.

9-13. (canceled)

14. The compound of claim 1, wherein if Xaa1 is absent, then the at least one residue pair is not Xaa2-Xaa6 or the N-terminus of the stapled peptide of formula (I) is not acylated.

15. The compound of claim 1, wherein amino acid residues in the at least one residue pair selected from Xaa1-Xaa5, Xaa2-Xaa6, Xaa2-Xaa9, Xaa5-Xaa9, Xaa5-Xaa12, Xaa6-Xaa10, and Xaa8-Xaa12 are selected from the group consisting of (S)-2-(4-pentenyl) alanine and (R)-2-(7-octenyl) alanine.

16. The compound of claim 1, which is selected from the group consisting of:
Xaa1 Thr Arg Arg Xaa5 Leu His Arg Ala Phe Asp Thr Leu Ala (SEQ ID NO:2),
Glu Xaa2 Arg Arg Met Xaa6 His Arg Ala Phe Asp Thr Leu Ala (SEQ ID NO:3),
Glu Thr Arg Arg Met Leu His Xaa8 Ala Phe Asp Xaa12 Leu Ala (SEQ ID NO:4),
Xaa1 Thr Arg Arg Xaa5 Leu His Xaa8 Ala Phe Asp Xaa12 Leu Ala (SEQ ID NO:5),
Xaa1 Thr Arg Arg Xaa5 Leu His Arg Ala Phe Asp Xaa12 Leu Ala (SEQ ID NO:6),
Xaa1 Glu Arg Arg Xaa5 Leu His Arg Xaa9 Phe Asp Thr Leu Ala (SEQ ID NO:7),
Glu Thr Arg Arg Xaa5 Leu His Arg Ala Phe Asp Xaa12 Leu Ala (SEQ ID NO:8),
Glu Thr Arg Arg Xaa5 Leu His Arg Xaa9 Phe Asp Thr Leu Ala (SEQ ID NO:9),
Glu Thr Arg Arg Met Xaa6 His Arg Ala Xaa10 Asp Thr Leu Ala (SEQ ID NO:10),
Glu Xaa2 Arg Arg Met Leu His Arg Xaa9 Phe Asp Thr Leu Ala (SEQ ID NO:11),
Glu Xaa2 Arg Arg Met Xaa6 His Xaa8 Ala Phe Asp Xaa12 Leu Ala (SEQ ID NO:12),
Ala Glu Xaa1 Thr Arg Arg Xaa5 Leu His Arg Ala Phe Asp Thr Leu Ala (SEQ ID NO:13),
Arg Arg Xaa5 Leu His Arg Ala Phe Asp Xaa12 Leu Ala (SEQ ID NO:14),
Xaa2 Arg Arg Met Xaa6 His Arg Ala Phe Asp Thr Leu Ala (SEQ ID NO:15),
Val Thr Arg Arg Xaa5 Leu His Arg Xaa9 Phe Asp Thr Leu Ala (SEQ ID NO:16),
Val Val Thr Arg Arg Xaa5 Leu His Arg Xaa9 Phe Asp Thr Leu Ala (SEQ ID NO:17),
Arg Thr Arg Arg Xaa5 Leu His Arg Xaa9 Phe Asp Thr Leu Ala (SEQ ID NO:18),
Arg Arg Thr Arg Arg Xaa5 Leu His Arg Xaa9 Phe Asp Thr Leu Ala (SEQ ID NO:19),
Ala Thr Arg Ala Xaa5 Leu His Arg Xaa9 Phe Asp Thr Leu Ala (SEQ ID NO:20),
Ala Thr Lys Ala Xaa5 Leu His Arg Xaa9 Phe Asp Thr Leu Ala (SEQ ID NO:21),
Ala Thr Arg Ala Met Xaa6 His Arg Ala Xaa10 Asp Thr Leu Ala (SEQ ID NO:22),
Ala Gly Ala Thr Ala Glu Glu Thr Arg Arg Xaa5 Leu His Arg Xaa9 Phe
Xaa1 Thr Arg Arg Xaa5 Leu His Arg Ala Phe Asp Thr Leu Ala (SEQ ID NO:23),
Ala Glu Xaa3 Thr Arg Arg Xaa7 Leu His Arg Ala Phe Asp Thr Leu Ala (SEQ ID NO:26),
Arg Arg Xaa3 Leu His Arg Ala Phe Asp Xaa10 Leu Ala (SEQ ID NO:27),
Glu Thr Arg Arg Xaa5 Leu His Arg Xaa9 Phe Asp Thr Leu Ala (SEQ ID NO:30),
Val Thr Arg Arg Xaa5 Leu His Arg Xaa9 Phe Asp Thr Leu Ala (SEQ ID NO:31),
Val Val Thr Arg Arg Xaa6 Leu His Arg Xaa10 Phe Asp Thr Leu Ala (SEQ ID NO:32),
Arg Thr Arg Arg Xaa5 Leu His Arg Xaa9 Phe Asp Thr Leu Ala (SEQ ID NO:33),
Arg Arg Thr Arg Arg Xaa6 Leu His Arg Xaa10 Phe Asp Thr Leu Ala (SEQ ID NO:34),
Ala Thr Arg Ala Xaa5 Leu His Arg Xaa9 Phe Asp Thr Leu Ala (SEQ ID NO:35),
Ala Thr Lvs Ala Xaa5 Leu His Arg Xaa9 Phe Asp Thr Leu Ala (SEQ ID NO:36),
Ala Thr Arg Ala Met Xaa6 His Arg Ala Xaa10 Asp Thr Leu Ala (SEQ ID NO:37),
Glu Thr Arg Arg Met Leu His Xaa8 Ala Phe Asp Xaa12 Leu Ala (SEQ ID NO:38),
Asp Thr Leu Ala (SEQ ID NO:40),
Phe Gly Ala Val Gly Ala Gly Ala Thr Ala Glu Glu Thr Arg Arg Xaa5
Leu His Arg Xaa9 Phe Asp Thr Leu Ala (SEQ ID NO:41),
Lys Thr Arg Arg Xaa5 Leu His Arg Xaa9 Phe Asp Thr Leu Ala (SEQ ID NO:42),
Gln Thr Arg Arg Xaa5 Leu His Arg Xaa9 Phe Asp Thr Leu Ala (SEQ ID NO:43),
Asn Thr Arg Arg Xaa5 Leu His Arg Xaa9 Phe Asp Thr Leu Ala (SEQ ID NO:44),
Val Val Val Thr Arg Arg Xaa5 Leu His Arg Xaa9 Phe Asp Thr Leu Ala (SEQ ID NO:45),
Ile Thr Arg Arg Xaa5 Leu His Arg Xaa9 Phe Asp Thr Leu Ala (SEQ ID NO:46),
Leu Thr Arg Arg Xaa5 Leu His Arg Xaa9 Phe Asp Thr Leu Ala (SEQ ID NO:47),
Phe Thr Arg Arg Xaa5 Leu His Arg Xaa9 Phe Asp Thr Leu Ala (SEQ ID NO:48),
Trp Thr Arg Arg Xaa5 Leu His Arg Xaa9 Phe Asp Thr Leu Ala (SEQ ID NO:49),
Tyr Thr Arg Arg Xaa5 Leu His Arg Xaa9 Phe Asp Thr Leu Ala (SEQ ID NO:50), and
Val Val Thr Arg Arg Cys Leu His Arg Cys Phe Asp Thr Leu Ala (SEQ ID NO:51).

17. (canceled)

18. (canceled)

19. A method of treating, ameliorating, and/or preventing HSV-1 propagation, processive DNA synthesis, and/or infection in a subject infected with HSV-1, the method comprising administering to the subject a therapeutically effective amount of the compound of claim 1.

20. (canceled)

21. A method of treating and/or preventing herpes keratitis in a subject, the method comprising administering to the subject a therapeutically effective amount of the compound of claim 1.

22. The method of claim 19, wherein at least one of the following applies:
(a) the compound is administered topically or ophthalmologically to the subject; and
(b) the compound is administered as part of a pharmaceutical composition.

23. (canceled)

24. The method of claim 19, wherein the subject is further administered an anti-herpetic agent, optionally wherein at least one of the following applies:
(a) the anti-herpetic agent is at least one selected from the group consisting of acyclovir, famciclovir, ganciclovir, penciclovir, valacyclovir, vidarabine, and trifluridine;
(b) the compound and the anti-herpetic agent are co-administered to the subject; and
(c) the compound and the anti-herpetic agent are co-formulated.

25-27. (canceled)

28. The method of claim 19, wherein the subject is a mammal, optionally wherein the mammal is a human.

29. (canceled)

30. A kit comprising the compound of claim 1, the kit further comprising an applicator; and an instructional material for the use of the kit, wherein the instruction material comprises instructions for treating, ameliorating, and/or preventing herpes keratitis in a subject, optionally wherein the kit further comprises an anti-herpetic agent.

31. (canceled)

32. A pharmaceutical composition comprising the compound of claim 1 and a pharmaceutically acceptable carrier.

33. The pharmaceutical composition of claim 32, wherein at least one of the following applies:
(a) the composition is formulated for topical administration; and
(b) the pharmaceutically acceptable carrier comprises liposomes, optionally wherein the liposomes are coated with chitosan.

34-35. (canceled)

36. The pharmaceutical composition of claim 32, wherein the compound is conjugated to a cyclic cell penetrating peptide, optionally wherein the cyclic cell penetrating peptide is CPP9.

37. (canceled)

38. The pharmaceutical composition of claim 36, wherein at least one of the following applies:
(a) the cyclic cell penetrating peptide is conjugated to the compound at the N-terminus or the C-terminus; and
(b) the cyclic cell penetrating peptide is conjugated to the compound via the linker.

39. (canceled)
